SEQUENCE LISTING (2 sequences)

SEQ ID NO: 1 - protein, 193 aa, Homo sapiens
MATPASAPDTRALVADFVGYKLRQKGYVCGAGPGEGPAADPLHQAMRAAGDEFETRFRRTFSDLAAQLHVTPGSAQQRFTQVSDELFQGGPNWGRLVAFFVFGAALCAESVNKEMEPLVGQVQEWMVAYLETRLADWIHSSGGWAEFTALYGDGALEEARRLREGNWASVRTVLTGAVALGALVTVGAFFASK

SEQ ID NO: 2 - protein, 183 aa, Artificial Sequence
  Bcl-w deltaC10 A128E
MATPASAPDTRALVADFVGYKLRQKGYVCGAGPGEGPAADPLHQAMRAAGDEFETRFRRTFSDLAAQLHVTPGSAQQRFTQVSDELFQGGPNWGRLVAFFVFGAALCAESVNKEMEPLVGQVQEWMVEYLETRLADWIHSSGGWAEFTALYGDGALEEARRLREGNWASVRTVLTGAVALGAL

(19) United States
(12) Patent Application Publication
(10) Pub. No.: US 2007/0054846 A1
(43) Pub. Date: Mar. 8, 2007

(54) Bcl-w structure and uses therefor

(76) Inventors: Mark Gavin Hinds, Victoria (AU); David Ching Siang Huang, Victoria (AU); Catherine Louise Day, Dunedin (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 10/537,635

(22) PCT Filed: Dec. 3, 2003
(86) PCT No.: PCT/AU03/01624

§ 371 Date: Jun. 29, 2006

(30) Foreign Application Priority Data
Dec. 3, 2002 (AU) 2002953259

(51) Int. Cl. A61K 38/17; G06F 19/00; G01N 33/574

(52) U.S. Cl. 514/12; 435/7.23; 530/350; 702/19

(57) ABSTRACT

The present invention discloses the solution structure of Bcl-w and Bcl-w complexes as well as methods of using that structural information to screen for and design compounds that interact with Bcl-w or variants thereof.

### FIELD OF THE INVENTION

THIS INVENTION relates generally to structural studies of a pro-survival protein. In particular, the present invention relates to the determination of the solution structure of Bcl-w including Bcl-w complexes. The invention also relates to methods of using the structural information to screen for and design compounds that interact with Bcl-w or variants thereof.

Bibliographic details of the publications referred to by author in this specification are collected at the end of the description.

### BACKGROUND OF THE INVENTION

Apoptosis, the physiological process of killing and removing damaged, unwanted or surplus cells during development, tissue homeostasis, or in response to stress or damage signals, is conserved between organisms as diverse as worms and man (Vaux and Korsmeyer, 1999). Since the deregulation of apoptosis has been linked to a number of diseased states, an understanding of how this process is controlled may allow novel ways to treat diseases, either by promoting or by inhibiting apoptosis (Thompson, 1995). For example, loss of myocardial tissues after acute myocardial infarcts may be limited by inhibiting apoptosis in the damaged tissues. Excessive apoptosis is also a feature of neuro-degenerative conditions such as Alzheimer's disease, suggesting that drugs preserving neuronal integrity may have a role in delaying the loss of vital neurones. In contrast to excess cell death, insufficient apoptosis is a feature of malignant disease and autoimmunity (Strasser et al, 1997). In either condition, persistence of damaged or unwanted cells that should normally be removed can contribute to disease.

In malignancies, mutations affecting cell death regulatory proteins or those that sense cellular damage have been described in various tumours. Bcl-2, the prototypic member of the Bcl-2 family of proteins, was cloned as the result of the t(14:18) chromosomal translocation in human follicular B cell lymphoma, which results in its overexpression (Tsujimoto et al, 1985; Cleary et al,

1986). Overexpression of Bcl-2, which functions to inhibit apoptosis (Vaux et al, 1988), or its functional homologues have also been reported in other tumours. However, mutations to proteins involved in sensing DNA damage are much more common in tumours. It is estimated that over half of human cancers have a mutation of the tumour suppressor protein, p53, or mutations affecting this pathway (Lane, 1992). p53 is necessary to elicit the appropriate cellular responses (growth arrest, apoptosis) to most forms of DNA damage. Interestingly, p53 kills cells mainly by a Bcl-2-dependent mechanism, since Bcl-2 overexpression can block most cell deaths induced by p53 (Lowe et al, 1993; Strasser et al, 1994). Both clinical observations and experiments in mouse models suggest that inhibition of apoptosis (e.g., p53 mutation, Bcl-2 overexpression) (Strasser et al, 1990; Adams et al, 1992) greatly promote oncogenic transformation caused by mutations that promote cellular proliferation alone (e.g., c-Myc overexpression, p21^{ras} mutations). Thus, reversing the process of tumorigenesis by promoting cell death, such as by activating p53 function or by inhibiting Bcl-2 function, may allow novel ways to complement the current treatments for malignancies. Furthermore, most of the cytotoxic treatments currently used to treat malignant diseases work partly by inducing the endogenous cell death machinery (Fisher, 1994), although this has been disputed by others (Brown and Wouters, 1999). For example, radiotherapy and many chemotherapeutic drugs activate apoptotic machinery indirectly by inducing DNA damage. Since the majority of tumours have mutations affecting the p53 pathway, many forms of therapy are significantly blunted because of the frequent loss of p53 function. The resistance of tumour cells to conventional agents provides further impetus to discovering novel cytotoxic drugs that act directly on the cell death machinery.

The effectors of cell death are cysteine proteases of the caspase family that cleave vital cellular substrates after aspartate residues (Thornberry, 1998). The caspases are synthesised as inactive zymogens and are only activated in response to cellular damage, thereby allowing exquisite control of apoptosis during normal tissue functioning in order to prevent inappropriate cell deaths. There are at least two distinct pathways to activate caspases in mammalian cells (Strasser et al, 2000). Binding of cognate ligands to some members of the TNF receptor superfamily induce cell death by activating the initiator caspase, caspase-8/FLICE, which is recruited to form oligomers on the receptor by the adaptor protein FADD/MORT-1 (Ashkenazi and Dixit, 1998). Once activated, caspase-8 can cleave downstream effector caspases such as caspases-3, -6, and -7, thereby massively amplifying the process.

A second pathway to caspase activation is that controlled by the Bcl-2 family of proteins (Adams and Cory, 2001). Overexpression of Bcl-2 can block many forms of physiologically (e.g., developmentally programmed cell deaths, death due to growth factor deprivation) and experimentally applied damage signals (e.g., cellular stress, radiation, most chemotherapeutic drugs). Bcl-2 controls the activation of the initiator caspase, caspase-9, by the adaptor protein Apaf-1, but this does not appear to be the critical or the sole molecule regulated by Bcl-2 (Moriishi et al, 1999; Conus et al, 2000; Hausmann et al, 2000; Haraguchi et al, 2000; Marsden et al., 2002). In the nematode C. elegans, the Bcl-2 homologue CED-9 functions by sequestering the activity of the adaptor protein CED-4 which is required to activate the caspase CED-3 (Spector et al, 1997; Chinnaiyan et al, 1997; Wu et al, 1997; Yang et al, 1998; Chen et al, 2000). However, a true mammalian homologue of CED-4 has not been discovered to date. The machinery is also more complex in mammals which express a number of structural and functional homologues of Bcl-2, namely Bcl-x_{L}, Bcl-w, Mcl-1 and A1 (Adams and Cory, 1998) (Cory and Adams, 2002). These pro-survival proteins are structurally similar, generally containing four conserved Bcl-2 homology domains (BH1-4), as well as a C-terminal hydrophobic region, promoting cell survival until antagonised by a family of distantly related proteins, the BH3-only proteins.

The BH3-only proteins are so-called because they share with each other, and with the other members of the Bcl-2 family of proteins, only the short BH3 domain (Huang and Strasser, 2000). This short domain forms an α-helical region, the hydrophobic face of which binds onto a hydrophobic surface cleft present on pro-survival Bcl-2 (Sattler et al, 1997; Petros et al, 2000). The BH3-only proteins probably function to sense cellular damage to critical cellular structures or metabolic process, and are then unleashed to initiate cell death by binding to and neutralising Bcl-2 (Huang and Strasser, 2000; Bouillet et al, 1999). Normally, the BH3-only proteins are kept inert by transcriptional or translational mechanisms, thereby preventing inappropriate cell deaths. Recently, two BH3-only proteins that are transcriptional targets of the tumour suppressor protein p53 have been described, namely Noxa (Oda et al, 2000) and Puma/Bbc3 (Yu et al, 2001; Nakano and Wousden, 2001; Han et al, 2001). These proteins are thus good candidates to mediate cell death induced by p53 activation (Vousden, 2000). Some other BH3-only proteins are controlled instead by post-translational mechanisms. In particular, two are sequestered to the cell's cytoskeletal network, Bim to the microtubules and Bmf to the actin cytoskeleton (Puthalakath et al, 1999; Puthalakath et al, 2001). Damage signals that impinge upon these cytoskeletal structures will activate Bim or Bmf freeing them to bind to pro-survival Bcl-2 located on the cytoplasmic face of the outer mitochondrial membrane as well as those of the nucleus and endoplasmic reticulum.

Recently it has been shown that the killing by the BH3-only proteins is dependent on the activity of a third family of Bcl-2-related proteins, namely the Bax sub-family (Zong et al., 2001; Cheng et al., 2001). Although these proteins bear three of the four homology domains and are structurally very similar to the pro-survival proteins (Suzuki et al, 2001), Bax, Bak and Bok/Mtd function instead to promote cell death. Biochemically, damage signals cause these proteins to aggregate and such oligomers may function to cause damage to mitochondrial membranes (Eskes et al., 2000; Desagher et al, 1999; Antonsson et al; 2001; Mikhailov et al., 2001; Wei et al., 2001; Jürgensmeier et al., 1998), thereby causing the release of mitochondrial pro-apoptogenic factors such as Smac/Diablo (Verhagen et al., 2000; Du et al., 2000) and cytochrome c, which is essential for the activation of caspase-9 by Apaf-1 (Kluck et al., 1997; Yang et al., 1997; Zou et al., 1997; Li et al., 1997). Since killing by BH3-only proteins depend on Bax and Bak in fibroblasts, their physiological role may be to activate Bax and Bak (Zong et al., 2001; Korsmeyer et al., 2000). In such a model, the pro-survival Bcl-2 proteins function merely to sequester the BH3-only proteins until such time as when there is insufficient capacity to do so. However, there are few reports of direct binding of the BH3-only proteins to Bax and Bak and even that in the case of the BH3-only protein Bid appears weak (Eskes et al., 2000; Wei et al., 2001; Wang et al., 1996). To date there are no experiments to directly compare the binding of BH3-only proteins with pro-survival Bcl-2 and to pro-apoptotic Bax.

In addition to the tenuous biochemical evidence for the direct binding of BH3-only proteins to Bax-like proteins, careful analyses of the available genetic data also suggests that pro-survival Bcl-2 rather than pro-apoptotic Bax is the direct target of BH3-only proteins. In the nematode C. elegans, all the killing induced by the BH3-only protein EGL-1 is dependent on the ability of EGL-1 to bind to and neutralise nematode Bcl-2, CED-9 (Conradt et al., 1998; Parrish et al., 2000). The situation is somewhat more complex in mammals because of the functional redundancy in each class of proteins. Instead of a single BH3-only protein (EGL-1) and a single Bcl-2 homologue (CED-9), mammals express multiple proteins of each sub-class making direct comparison with the nematode difficult. Furthermore, nematodes do not appear to express Bax-like proteins. However, if the Bcl-2-like proteins function merely to sequester BH3-only proteins, then the amount of pro-survival Bcl-2-like proteins in any cell type must be limiting. It is therefore surprising that mice lacking a single allele of the bcl-2 (Veis et al., 1993; Nakayama et al., 1994; Kamada et al., 1995), bcl-x (Motoyama et al., 1995; Motoyama et al., 1999) or bcl-w (Ross et al., 1998; Print et al., 1998) genes are normal whereas the homozygous knock-out mice all have striking phenotypes in the cell types where these genes play a crucial role. This suggests that the pro-survival Bcl-2-like proteins are not limiting; instead analysis of mice lacking the BH3-only protein Bim suggest that this class of proteins is limiting (Bouillet et al., 1999; Bouillet et al.,

2001). Taken together, the available data would suggest that BH3-only proteins directly bind to Bcl-2 and it is by neutralising Bcl-2 that BH3-only proteins can activate Bax-like proteins.

Thus, agents that directly mimic the BH3-only proteins may induce cell death and may, therefore, be of value therapeutically. As Bcl-2 controls the critical point that determines a cell's fate, this class of proteins represent an attractive target for drug design. In particular, since many of the oncogenic mutations, such as those to p53 results in defects in sensing cellular damage that would normally result in cell death by a Bcl-2-dependent mechanism, directly targeting Bcl-2 and its homologues may circumvent such mutations. This may also permit an alternative route to overcome tumour resistance to current treatments.

A general approach to designing drugs that are selective for a target protein is to determine how a putative drug interacts with the three dimensional structure of that protein. For this reason it is useful to determine the three dimensional structure (coordinates) of a target protein and preferably the target protein in complex with a cognate ligand. From the latter structure, one can determine both the shape of the protein's binding pocket when bound to the ligand, as well as the amino acid residues that are capable of close contact with the ligand. By having knowledge of the shape and amino acid residues in the binding pocket, one may design new ligands that will interact favourably with the protein. With such structural information, available computational methods may be used to predict the strength of the ligand-binding interaction. Such methods thus enable the design of drugs (e.g., agonists or antagonists) that bind strongly, as well as selectively to the target protein.

Accordingly, knowledge of the three-dimensional structure of Bcl-2 proteins and its homologues would be useful in facilitating the design of antagonists of these proteins, which, in turn, are expected to have therapeutic utility. In this regard, solution structures of Bcl-x_{L} (Muchmore et al., 1996), Bcl-2 (Petros et al., 2001), and the Kaposi Sarcoma Herpes Virus (KSHV) Bcl-2 homologue (Huang et al., 2002), reveal that the BH1-3 domains are in close proximity to each other and form a hydrophobic groove that is the docking site for BH3-only proteins (Petros et al., 2000; Sattler et al., 1997). However, in contrast to the structures of C-terminally truncated Bcl-x_{L} or Bcl-2, the hydrophobic groove formed by the BH1-3 domains in Bax is occluded by its C-terminus. Translocation of Bax from the cytosol to intracellular membranes, particularly the outer mitochondrial membrane (Nechushtan et al., 1999; Nechushtan et al., 2001), is an early step in its damage signal induced activation and exposure of the hydrophobic C-terminus may be important to this process.

Although pro-survival Bcl-w is functionally indistinguishable from Bcl-2 and Bcl-x_{L}(Gibson et al., 1996; O'Reilly et al., 2001), it appears to be located exclusively on the outer mitochondrial membrane, whereas a significant proportion of Bcl-2 (˜90%) (Krajewski et al., 1993; Lithgow et al., 1994) and Bcl-x_{L} (˜50%) (Gonzalez-Garcia et al., 1994; Hsu et a, 1997) is present on the outer nuclear and contiguous endoplasmic membranes. While associated with mitochondria in healthy cells, Bcl-w is only weakly attached to the membranes. However, binding of a BH3-only protein, such as Bim activated by death signals, triggers tight membrane association of Bcl-w in dying cells. A likely explanation for the tighter membrane association upon binding of a BH3-only protein is that a conformational change occurs, exposing the C-terminus of Bcl-w, thereby allowing it to interact with the mitochondrial membrane. However, no such change was apparent from the structures of C-terminally truncated Bcl-x_{L} in complex with either Bak or Bad BH3 peptides (Petros et al., 2000; Sattler et al., 1997). Instead, the hydrophobic C-terminal residues that are present are not well structured and make no contacts with the body of the protein. Furthermore, it appears that the BH3-binding groove on pro-survival molecules pre-exists and ligand binding does not cause major conformational alteration.

### SUMMARY OF THE INVENTION

The present invention is predicated in part on the three-dimensional structure of a Bcl-w derivative and of certain Bcl-w-ligand complexes and more specifically, on their solution structures, as determined using spectroscopy and various computer modelling techniques. The key structural features of Bcl-w revealed thereby, particularly the shape, architecture and physicochemical properties of the active site in which BH3-only proteins bind, are useful for identifying, selecting or designing agents that are capable of inhibiting or potentiating at least one biological activity of Bcl-w and in solving the structures of other proteins with similar structures, as described hereafter.

Thus, in one aspect of the present invention, there is provided a solution comprising a molecule or molecular complex that comprises a Bcl-w active site as herein defined. Preferably, the molecule or molecular complex further comprises the C-terminal region of Bcl-w, which suitably comprises the C-terminal helix (α9, residues 157-173) and extended region (residues 174-183) of Bcl-w.

Suitably, the molecule or molecular complex comprises a polypeptide that is distinguished from Bcl-w by the deletion of at least one amino acid at the C-terminus of Bcl-w. In one embodiment of this type, the polypeptide is distinguished from Bcl-w by the deletion of at least five amino acid residues, and more preferably by 10 amino acid residues, from the C-terminus of Bcl-w.

Preferably, the polypeptide is further distinguished from Bcl-w by the substitution of at least one hydrophobic amino acid residue with a charged amino acid residue. In a preferred embodiment of this type, the hydrophobic amino acid residue is Ala 128 and the charged amino acid residue is glutamate or modified form thereof.

In an especially preferred embodiment, the polypeptide comprises the sequence set forth in SEQ ID NO:2, which defines a Bcl-w derivative that lacks the last 10 amino acid residues of Bcl-w and that has Ala128 substituted with a glutamate residue or modified form thereof. The three dimensional solution structure of this polypeptide, hereafter referred to as Bcl-wΔC10, is provided by the relative atomic structural coordinates of TABLE 1, as obtained from spectroscopy data.

In another aspect, the present invention provides a polypeptide as broadly defined above.

In other aspects, the present invention extends to polynucleotides that encode the polypeptide as broadly defined above, to vectors comprising those polynucleotides and to hosts cells containing such vectors.

The solution coordinates of Bcl-wΔC10 or portions thereof (such as the Bcl-w active site as herein defined), as provided by this invention may be stored in data store such as in a machine-readable form on a machine-readable storage medium, e.g. a computer hard drive, diskette, DAT tape, etc., for display as a three-dimensional shape or for other uses involving computer-assisted manipulation of, or computation based on, the structural coordinates or the three-dimensional structures they define. By way of example, the data defining the three dimensional structure of a Bcl-w derivative as set forth in TABLE 1 may be stored in a machine-readable storage medium, and may be displayed as a graphical three-dimensional representation of the relevant structural coordinates, typically using a computer capable of reading the data from said storage medium and programmed with instructions for creating the representation from such data. Accordingly, the present invention embraces a machine, such as a computer, programmed in memory with the coordinates of a Bcl-w derivative or portions thereof, together with a program capable of converting the coordinates into a three dimensional graphical representation of the structural coordinates on a display connected to the machine. A machine having a memory containing such data aids in the rational design or selection of agonists or antagonists of Bcl-w binding or activity, including the evaluation of the ability of a particular chemical entity to favourably associate with Bcl-w as disclosed herein, as well as in the modelling of compounds, proteins, complexes, etc. related by structural or sequence homology to Bcl-w.

Thus, in yet another aspect of the present invention, there is provided a data store comprising data representing the structure coordinates of Bcl-w amino acid residues and which are capable of being used by a computer system to generate a three-dimensional representation of a molecule or molecular complex comprising a Bcl-w active site defined by the structure coordinates of at least three Bcl-w amino acid residues selected from Arg59, Asp63, Leu64, Gln67, Phe79, Val82, Val102 and Leu106 as set forth in TABLE 1, or a variant of the molecule or molecular complex, wherein the variant comprises an active site that has a root mean square deviation from the Cα atoms of the amino acid residues defining the Bcl-w active site of not more than 1.1 Å.

Preferably, the active site is further defined by the structure coordinates of at least three Bcl-w amino acid residues selected from Glu52, Arg56, Arg58, Glu85, Arg95 and Lys113 as set forth in TABLE 1.

In a preferred embodiment, the active site is defined by the structure coordinates of at least three Bcl-w amino acid residues, which are within 5 Å of the C-terminal region of Bcl-w, including but not limited to, Gln44, Ala45, Ala48, Ala49, Gly50, Glu52, Phe53, Arg56, Phe57, Arg58, Arg59, Asp63, Leu64, Ala66, Gln67, His69, Val70, Arg78, Phe79, Gln81, Val82, Ser83, Glu85, Leu86, Phe87, Gln88, Gly89, Gly90, Pro91, Asn92, Trp93, Gly94, Arg95, Val97, Phe99, Phe102, Leu106, Phe147, Thr148, Ala149, Leu150, Tyr151 and Gly152, as set forth in TABLE 1.

In another preferred embodiment, the active site is defined by the structure coordinates of at least three Bcl-w amino acid residues, which are within 8 Å of the C-terminal region of Bcl-w, including but not limited to, Gln44, Ala45, Met46, Arg47, Ala48, Ala49, Gly50, Asp51, Glu52, Phe53, Glu54, Thr55, Arg56, Phe57, Arg58, Arg59, Thr60, Ser62, Asp63, Leu64, Ala65, Ala66, Gln67, Leu68, His69, Val70, Thr71, Ala75, Gln76, Gln77, Arg78, Phe79, Thr80, Gln81, Val82, Ser83, Asp84, Glu85, Leu86, Phe87, Gln88, Gly89, Gly90, Pro91, Asn92, Trp93, Gly94, Arg95, Leu96, Val97, Ala98, Phe99, Phe102, Gly103, Leu106, Trp137, Ser141, Glu146, Phe147, Thr148, Ala149, Leu150, Tyr151 and Gly152, as set forth in TABLE 1.

In another aspect, the invention provides a computer system having data representing structural coordinates of Bcl-w amino acid residues, the computer system being adapted to generate, on the basis of the data, a three-dimensional representation of a molecule or molecular complex comprising a Bcl-w active site as defined above, or a variant of the molecule or molecular complex, wherein the variant comprises an active site that has a root mean square deviation from the Cα atoms of the amino acid residues defining the Bcl-w active site of not more than 1.1 Å.

In yet another aspect, the invention provides a computer system for producing a three-dimensional representation of a molecule or molecular complex, the computer system comprising: (a) a data store including data representing the structure coordinates of Bcl-w amino acid residues defining a Bcl-w active site of the present invention, or structural coordinates having a root mean square deviation from the Cα atoms of those residues of not more than 1.1 Å; (b) a processing means for processing the data to generate a three-dimensional representation of a molecule or molecular complex comprising the Bcl-w active site or similarly shaped homologous active site for display; and (c) a display means for displaying the three-dimensional representation.

In still another aspect, the invention provides an analysis method, executed by a computer system, for evaluating the ability of a chemical entity to associate with a molecule or molecular complex comprising an active site, the method comprising the steps of: (a) generating a model of the active site using structure coordinates wherein the root mean square deviation between the structure coordinates and the structure coordinates of the Bcl-w amino acid residues defining a Bcl-w active site of the invention is not more than about 1.1 Å; (b) performing a fitting operation between the chemical entity and the model of the active site; and (c) quantifying the association between the chemical entity and the active site model, based on the output of the fitting operation.

In a further aspect of the invention, there is provided an analysis method, executed by a computer system, for comparing the ability of a chemical entity to associate with a first molecule or molecular complex comprising a first active site and the ability of the chemical entity to associate with a second molecule or molecular complex comprising a second active site, the method comprising the steps of: (a) generating a model of the first active site using structure coordinates wherein the root mean square deviation between the structure coordinates and the structure coordinates of the Bcl-w amino acid residues defining a Bcl-w active site of the invention is not more than about 1.1 Å; (b) performing a first fitting operation between the chemical entity and the model of the first active site; (c) quantifying the association between the chemical entity and the first active site model, based on the output of the first fitting operation; (d) performing a second fitting operation between the chemical entity and a model of the second active site; (e) quantifying the association between the chemical entity and the second active site model, based on the output of the second fitting operation; and (f) comparing the respective associations of the chemical entity with the first active site model and with the second active site model.

In one embodiment of this type, the second molecule or molecular complex comprises an active site of another pro-survival protein such as but not limited to Bcl-2, Bcl-x_{L}, Mcl-1 and A1, or variant thereof.

In yet a further aspect of the invention, there is provided an analysis method, executed by a computer system, for identifying a chemical entity that associates with both a first molecule or molecular complex comprising a first active site and a second molecule or molecular complex comprising a second active site, the method comprising the steps of: (a) generating a model of the first active site using structure coordinates wherein the root mean square deviation between the structure coordinates and the structure coordinates of the Bcl-w amino acid residues defining a Bcl-w active site of the invention is not more than about 1.1 Å; (b) performing a fitting operation between the chemical entity and the model of the first active site; (c) quantifying the association between the chemical entity and the first active site model, based on the output of the first fitting operation; (d) performing a second fitting operation between the chemical entity and a model of the second active site; (e) quantifying the association between the chemical entity and the second active site model, based on the output of the second fitting operation; and (f) comparing the respective associations of the chemical entity with the first active site model and with the second active site model to determine whether the chemical entity associates individually with both the first molecule or molecular complex and the second molecule or molecular complex.

In still a further aspect of the invention, there is provided an analysis method, executed by a computer system, for identifying a chemical entity that associates more favourably with a first molecule or molecular complex comprising a first active site than with a second molecule or molecular complex comprising a second active site, the method comprising the steps of: (a) generating a model of the first active site using structure coordinates wherein the root mean square deviation between the structure coordinates and the structure coordinates of the Bcl-w amino acid residues defining a Bcl-w active site of the invention is not more than about 1.1 Å; (b) performing a fitting operation between the chemical entity and the model of the first active site; (c) quantifying the association between the chemical entity and the first active site model, based on the output of the first fitting operation; (d) performing a second fitting operation between the chemical entity and a model of the second active site; (e) quantifying the association between the chemical entity and the second active site model, based on the output of the second fitting operation; and (f) comparing the respective associations of the chemical entity with the first active site model and with the second active site model to determine whether the chemical entity associates more favourably with the first molecule or molecular complex than with the second molecule or molecular complex.

In still a further aspect, the invention encompasses a method for identifying a potential antagonist of a molecule comprising a Bcl-w-like active site, comprising the steps of: (a) generating a three-dimensional structure of the molecule comprising the active site using the atomic coordinates of at least three Bcl-w amino acid residues selected from Arg59, Asp63, Leu64, Gln67, Phe79, Val82, Val102 and Leu106 as set forth in TABLE 1 ± a root mean square deviation from the Cαatoms of those residues of not more than 1.1 Å; (b) employing the three-dimensional structure to identify, design or select the potential antagonist; (c) synthesising or otherwise obtaining the antagonist; and (d) contacting the antagonist with the molecule to determine the ability of the potential antagonist to interact with said molecule.

In a preferred embodiment of this type, the three-dimensional structure of the molecule comprising the active site is generated further using structure coordinates of at least three Bcl-w amino acid residues selected from Glu52, Arg56, Arg58, Asp63, Glu85, Arg95 and Lys113 as set forth in TABLE 1 ± a root mean square deviation from the Cα atoms of those residues of not more than 1.1 Å.

In another preferred embodiment of this type, the three-dimensional structure of the molecule comprising the active site is generated further using structure coordinates of at least three Bcl-w amino acid residues, which are within 8 Å of the C-terminal region of Bcl-w, as for example defined above.

In yet another preferred embodiment of this type, the three-dimensional structure of the molecule comprising the active site is generated further using structure coordinates of at least three Bcl-w amino acid residues, which are within 8 Å of the C-terminal region of Bcl-w, as for example defined above.

In an even more preferred embodiment, the three-dimensional structure of the molecule comprising the active site is created using the structure coordinates of all the Bcl-w amino acid residues as set forth in TABLE 1 ± a root mean square deviation from the Cα atoms of those residues of not more than 1.1 Å.

The antagonist may be selected by screening an appropriate database, may be designed de novo by analysing the steric configurations and charge potentials of an empty Bcl-w active site in conjunction with the appropriate software programs, or may be designed using characteristics of known antagonists to create “hybrid” antagonists. The antagonist may then be contacted with Bcl-w, or a Bcl-w derivative, alone (using Bcl-w or a molecule comprising a Bcl-w active site such as Bcl-wΔC10), or in the presence of a BH3 ligand such as Bim BH3 as described infra, and the effect of the antagonist on Bcl-w or Bcl-w derivative alone or binding between Bcl-w and the BH3 ligand may be assessed. It is also within the confines of the present invention that a potential antagonist may be designed or selected by identifying chemical entities or fragments capable of associating with Bcl-w; and assembling the identified chemical entities or fragments into a single molecule to provide the structure of the potential inhibitor.

In still yet another aspect, the present invention provides agents or antagonists designed or selected using the methods disclosed herein.

A further aspect of the present invention provides a method for determining at least a portion of the three-dimensional structure of other molecules or molecular complexes which contain at least some features that are structurally similar to Bcl-w by using at least some of the structural coordinates obtained for Bcl-w. This method comprises the steps of first obtaining crystals or a solution of the molecule or molecular complex whose structure is unknown, and then generating X-ray diffraction data from the crystallised molecule or molecular complex and/or generating NMR data from the solution of the molecule or molecular complex. The generated diffraction or spectroscopy data from the molecule or molecular complex can then be compared with the solution coordinates or three dimensional structure of Bcl-w derivative as disclosed herein, and the three dimensional structure of the unknown molecule or molecular complex conformed to the Bcl-w derivative structure using standard techniques such as molecular replacement analysis, 2D, 3D and 4D isotope filtering, editing and triple resonance NMR techniques, and computer homology modelling. Alternatively, a three dimensional model of the unknown molecule may be generated by generating a sequence alignment between Bcl-w derivative and the unknown molecule, based on any or all of amino acid sequence identity, secondary structure elements or tertiary folds, and then generating by computer modelling a three dimensional structure for the molecule using the three dimensional structure of, and sequence alignment with, the Bcl-w derivative.

### BRIEF DESCRIPTION OF THE DRAWINGS

FIG. 1 is a diagrammatic representation showing the sequence and structure of Bcl-w. FIG. 1A illustrates a stereoview of the backbone (N, Cα, C) superposition of the 20 NMR derived structures of Bcl-wΔC10 (residues 8-183). Aromatic side chains are shown in different colours: Trp (green), Phe (red), His (cyan) and Tyr (yellow). The region of extended structure at the C-terminus is shown in purple. FIG. 1B is a ribbon diagram of the structure closest to the mean (residues 8-183). The helices are indicated in different colours and are labelled. The view on the left has the same orientation as FIG. 1A while the middle view has been rotated 180° about the vertical axis and the right view 90° about the horizontal axis. FIG. 1C shows a structure-based sequence alignment of Bcl-w, Bcl-x_{L}, Bcl-2 and Bax. The Bcl-2 homology (BH) domains are indicated by the bars above the sequences and the limits of the secondary structure are depicted by the coloured boxes within the sequence and named (α1-α9) beneath them. Residue numbers above the sequences refer to Bcl-w. * residues of Bcl-w whose HN protons are in fast exchange with the solvent.

FIG. 2 is a diagrammatic representation showing the hydrophobic binding grooves in Bcl-2 family members. FIG. 2A is a close-up view of the C-terminal residues of Bcl-w. Residues 8-152 are shown as a surface with the side chains of basic, acidic and hydrophobic residues coloured blue, red and yellow, respectively. The C-terminal residues (153-183) are shown as a ribbon (purple) and the side chains of these residues are shown in stick representation (green). FIG. 2B illustrates a comparison of the hydrophobic binding grooves from Bcl-w, Bax and Bcl-x_{L}. In all three structures residues equivalent to 8-152 in Bcl-w are shown as a surface representation with the BH domains indicated (BH1green; BH2 pink; BH3 yellow). The residues that lie in the groove (Bcl-w residues 153-181, Bax residues 166-192 and the Bad peptide) are shown as a ribbon (light blue) with the side chains as sticks (blue). The atomic coordinates of Bax (1f16) and the Bcl-x_{L}Bad (1g5j) peptide complex were obtained form the Protein Data Bank. FIG. 2C depicts a comparison of the binding groove in Bcl-w with those in Bax and Bcl-x_{L}. On the left, the ribbon diagram representing Bcl-w (pale blue) is superimposed with Bax (yellow). The C-terminal residues are shown in dark blue (Bcl-w) and dark yellow (Bax). On the right Bcl-w (pale blue, dark blue) is superimposed with Bcl-x_{L} (pink):Bad (dark pink) complex. The structures were superimposed using TOP (Lu, 2000) and the equivalent view is shown for all of them.

FIG. 3 comprises tabular, graphical and photographic representations showing the binding properties of Bcl-w proteins. FIG. 3A is a table showing the binding constants for various Bcl-w-BH3-only ligand complexes, which were determined using Biosensor experiments as described herein. FIG. 3B is a graphical representation showing the interaction kinetics of Bcl-w binding to Bim_{L}ΔC27. Samples of serially diluted Bcl-w (2 μM-62.5 nM) were analysed on a Bim_{L}ΔC27 sensor surface as described in Experimental Procedures. The experimental data (−) and the suggested fit to a 1:1 Langmuir binding model (•••) are illustrated. FIG. 3C is a graphical representation showing the interaction kinetics of Bcl-w or Bcl-wΔC29 binding to Bim_{L}ΔC27 or Bim_{L}ΔC27-L94A. Serial dilutions of Bcl-w or Bcl-wΔC29 were analysed on parallel sensor surfaces that had been derivatised at comparable densities with either Bim_{L}ΔC27 or Bim_{L}ΔC27-L94A. Relative responses of samples between 1 μM and 62.5 nM are shown. FIG. 3D is a photographic representation of a GST pull-down assay to assess the binding capacity of Bcl-w proteins. Approximately equivalent amounts of the indicated GST-Bcl-w proteins were mixed with either soluble wt Bim_{L}ΔC27 or soluble Bim_{L}ΔC27-L94A. The intensity of the Bim band indicated the amount of protein that bound to Bcl-w. Molecular weight standards in kDa are indicated.

FIG. 4 comprises photographic representations showing that the in vivo and in vitro binding properties of Bcl-x_{L} resemble that of Bcl-w. FIG. 4A is a photographic representation showing that the C-terminus of Bcl-w restricts access to the binding groove in vivo. Equivalent ³⁵S-labeled 293T lysates obtained from cells co-expressing FLAG Bcl-w or Bcl-wΔC29, and EE-Bim_{EL} or Bim_{EL}-L150A, were immunoprecipitated using the anti-FLAG M2 (α-F), anti-EE (α-E) or control anti-HA (α-H) monoclonal antibodies. The immunoprecipitations were fractionated on SDS-PAGE gels. FIG. 4B is a photographic representation showing that the C-terminus of Bcl-x_{L} restricts access to the binding groove in vivo. Co-precipitation experiments similar to those described in FIG. 4A using lysates from cells co-expressing FLAG Bcl-x_{L} or Bcl-x_{L}ΔC24, and EE-Bim_{L} or Bim_{L}-L94A. FIG. 4C is a photographic representation showing that a GST pull-down experiment as for FIG. 3D, except in this case GST-Bcl-x_{L} proteins were mixed with either soluble wt Bim_{L}ΔC27 or soluble Bim_{L}ΔC27-L94A. The intensity of the Bim band indicated the amount of protein that bound to Bcl-x_{L}.

FIG. 5 contains graphical and tabular illustrations showing that Bcl-wΔC10 is functionally inert but is structurally similar to biologically active Bcl-wΔC5. FIG. 5A is a graphical representation showing that Bcl-w cannot tolerate extensive C-terminal deletions. The viability of parental FDC-P1 cells (□) or representative clones expressing different Bcl-w constructs (Bcl-w ●; Bcl-w (A128E)♦; Bcl-wΔC3 ▴; Bcl-wΔC5 ▾; Bcl-wΔC10 ⋄; Bcl-wΔC23 Δ; Bcl-wΔC29 ◯) deprived of IL-3 were determined by PI staining analysed flow cytometrically. Data shown are means +/−1 SD of at least 3 experiments. FIG. 5B is a table summarising the binding properties and biological activity of full-length or C-terminal truncated mutants of Bcl-w. FIG. 5A is a graphical representation showing a comparison of the 2D ¹H-¹⁵N-HSQC spectra for Bcl-wΔC10 and Bcl-wΔC5. Backbone amide chemical shift differences plotted for residues in ¹⁵N labelled Bcl-wΔC10 relative to those for Bcl-wΔC5 are indicated. Colours for the helices correspond to those used in FIG. 1.

FIG. 6 is a diagrammatic representation showing residues Ala49, Gly50, Asp51, Phe53, Arg58, Phe61, Asp63, Leu64, Ala66, His69, Thr71, Ala75, Phe79, Ser83, Gln88, Asn92, Trp93, Gly94, Val101, F102, Gly103, Glu114, Gly120, Gln121, Gln123, Leu134, Ala135, Trp144, Phe147, Thr148, Ala149, Tyr151, Glu158, Ala159, Arg160, Leu162, Arg163, Asn166, Trp167, Ala168, Ser169, Val170, Thr172, Val173, Leu174, Thr175, Gly176, Ala177, Val178, Ala179 (in orange) on Bcl-wΔC10 whose resonances shift in a ¹⁵N-NOESY-HSQC on addition of Bim-BH3 peptide.

FIG. 7 is a diagrammatic representation of the charge distribution on Bcl-wΔC41. Electrostatic charge was calculated in Delphi, simple charge with backbone atoms partially charged (HN, N, O, CA, C') as per the GRASP manual. Levels >+8 kT (blue), <−8 kT (red). F57 cyan and the L 180 cavity labelled.

FIG. 8 is a schematic representation of a computer system useful in the practice of the present invention.

TABLE A
BRIEF DESCRIPTION OF THE SEQUENCES

| SEQUENCE ID | | |
|---|---|---|
| NUMBER | SEQUENCE | LENGTH |
| SEQ ID NO: 1 | Amino acid sequence of wild-type Bcl-w | 193 aa |
| SEQ ID NO: 2 | Amino acid sequence of Bcl-wΔC10 | 183 aa |

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “about” is used herein to refer to values or amounts that vary by as much as 30%, preferably by as much as 20%, and more preferably by as much as 10% to a reference value or amount.

The term “active site” refers to a region of a molecule or molecular complex that, as a result of its shape and charge potential, favourably interacts or associates with another agent (including, without limitation, a protein, polypeptide, peptide, nucleic acid, including DNA or RNA, molecule, compound, antibiotic or drug) via various covalent and/or non-covalent binding forces. As such, an active site of the present invention may include, for example, the actual site of Bcl-w binding with a BH3 ligand, as well as accessory binding sites adjacent to the actual site of BH3 ligand binding that nonetheless may affect Bcl-w upon interaction or association with a particular agent (e.g., sites that interact with the C-terminal region of Bcl-w), either by direct interference with the actual site of BH3 ligand binding or by indirectly affecting the steric conformation or charge potential of Bcl-w and thereby preventing or reducing BH3 ligand binding to Bcl-w at the actual site of BH3 ligand binding. As used herein, “active site” also includes any Bcl-w site of self association, as well as other binding sites present on Bcl-w.

The term “agonist” refers to a ligand that when bound to a pro-survival protein, especially a Bcl-w protein or variant or derivative thereof, stimulates its activity.

The term “altered surface charge” means a change in one or more of the charge units of a variant polypeptide, at physiological pH, as compared to wild-type Bcl-w. This is preferably achieved by replacement of at least one amino acid of wild-type Bcl-w with another amino acid comprising a side chain with a different charge at physiological pH than the original wild-type side chain. The change in surface charge is suitably determined by measuring the isoelectric point (pI) of the polypeptide molecule containing the substituted amino acid and comparing it to the isoelectric point of the wild-type Bcl-w molecule.

The term “antagonist” refers to a ligand that when bound to a pro-survival protein, especially a Bcl-w protein or variant or derivative thereof, inhibits its activity.

The term “associating with” refers to a condition of proximity between a chemical entity or compound, or portions thereof, and a Bcl-w molecule or portions thereof. The association may be non-covalent—wherein the juxtaposition is energetically favoured by hydrogen bonding or van der Waals or electrostatic interactions—or it may be covalent.

The term “β-sheet” refers to the conformation of a polypeptide chain stretched into an extended zigzag conformation. Portions of polypeptide chains that run “parallel” all run in the same direction. Polypeptide chains that are “antiparallel” run in the opposite direction from the parallel chains.

A “Bcl-w complex” refers to a co-complex of a molecule comprising a Bcl-w active site in bound association with a protein, polypeptide, peptide, nucleic acid, including DNA or RNA, small molecule, compound or drug, either by covalent or non-covalent binding forces. A non-limiting example of a Bcl-w complex includes Bcl-w or a Bcl-w variant bound to a BH3 ligand.

The term “Bcl-w-like active site ” and the like refers to a portion of a molecule or molecular complex whose shape is sufficiently similar to all or any parts of the active site of Bcl-w as to bind common ligands. This commonality of shape is defined by a root mean square deviation (rmsd) from the structure coordinates of the Cα atoms of the amino acid residues that make up the active site in Bcl-w (as set forth in TABLE 1) of not more than 1.1 Å. How this calculation is obtained is described below. More preferably, the root mean square deviation is less than about 1.0 Å.

The term “chemical entity”, as used herein, refers to chemical compounds or ligands, including proteins, polypeptides, peptides, nucleic acids, including DNA or RNA, molecules, or drugs, complexes of at least two chemical compounds, and fragments of such compounds or complexes.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions, or deletions that provide for functionally equivalent molecules.

The term “hydrophobic amino acid” means any amino acid having an uncharged, non-polar side chain that is relatively insoluble in water. Examples of naturally occurring hydrophobic amino acids are alanine, leucine, isoleucine, valine, proline, phenylalanine, tryptophan and methionine.

The term “hydrophilic amino acid” means any amino acid having an uncharged, polar side chain that is relatively soluble in water. Examples of naturally occurring hydrophilic amino acids are serine, threonine, tyrosine, asparagine, glutamine, and cysteine.

The term “naturally occurring amino acids” means the L-isomers of the naturally occurring amino acids. The naturally occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, α-carboxyglutamic acid, arginine, ornithine and lysine. Unless specifically indicated, all amino acids referred to in this application are in the L-form.

The term “negatively charged amino acid” includes any naturally occurring or unnatural amino acid having a negatively charged side chain under normal physiological conditions. Examples of negatively charged naturally occurring amino acids are aspartic acid and glutamic acid.

The term “polynucleotide” or “nucleic acid” as used herein designates mRNA, RNA, cRNA, cDNA or DNA. The term typically refers to oligonucleotides greater than 30 nucleotides in length.

The terms “polynucleotide variant” and “variant” refer to polynucleotides displaying substantial sequence identity with a reference polynucleotide sequence or polynucleotides that hybridise with a reference sequence under stringent conditions that are defined hereinafter. These terms also encompass polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. The terms “polynucleotide variant” and “variant” also include naturally occurring allelic variants. The term “variant” refers to a protein having at least 30% amino acid sequence identity with Bcl-w or any functional domain of Bcl-w, including its active site and C-terminal region.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

The term “polypeptide variant” refers to polypeptides that vary from a reference polypeptide by the addition, deletion or substitution of at least one amino acid. It is well understood in the art that some amino acids may be changed to others with broadly similar properties without changing the nature of the activity of the polypeptide (conservative substitutions) as described hereinafter. Accordingly, polypeptide variants as used herein encompass polypeptides that have pro-survival activity. The term “variant” refers to a protein having at least 30% amino acid sequence identity with a reference protein or any functional domain thereof. More specifically, the term “variant” includes, but is not limited to, a polypeptide comprising an active site characterised by a three dimensional structure comprising (i) the relative structural coordinates of at least three Bcl-w amino acid residues selected from Arg59, Asp63, Leu64, Gln67, Phe79, Val82, Val102 and Leu106 as set forth in TABLE 1, (ii) the relative structural coordinates of amino acid Glu52, Arg56, Arg58, Asp63, Glu85, Arg95 and Lys113 as set forth in TABLE 1, (iii) the relative structural coordinates of at least three Bcl-w amino acid residues selected from Gln44, Ala45, Ala48, Ala49, Gly50, Glu52, Phe53, Arg56, Phe57, Arg58, Arg59, Asp63, Leu64, Ala66, Gln67, His69, Val70, Arg78, Phe79, Gln81, Val82, Ser83, Glu85, Leu86, Phe87, Gln88, Gly89, Gly90, Pro91, Asn92, Trp93, Gly94, Arg95, Val97, Phe99, Phe102, Leu106, Phe147, Thr148, Ala149, Leu150, Tyr151 and Gly152, as set forth in TABLE 1, or (iv) the relative structural coordinates of at least three Bcl-w amino acid residues selected from Gln44, Ala45, Met46, Arg47, Ala48, Ala49, Gly50, Asp51, Glu52, Phe53, Glu54, Thr55, Arg56, Phe57, Arg58, Arg59, Thr60, Ser62, Asp63, Leu64, Ala65, Ala66, Gln67, Leu68, His69, Val70, Thr71, Ala75, Gln76, Gln77, Arg78, Phe79, Thr80, Gln81, Val82, Ser83, Asp84, Glu85, Leu86, Phe87, Gln88, Gly89, Gly90, Pro91, Asn92, Trp93, Gly94, Arg95, Leu96, Val97, Ala98, Phe99, Phe102, Gly103, Leu106, Trp137, Ser141, Glu146, Phe147, Thr148, Ala149, Leu150, Tyr151 and Gly152, as set forth in TABLE 1, in each case, ±a root mean square deviation from the conserved backbone atoms of those residues of not more than 1.1 Å, more preferably not more than 1.0 Å, and most preferably not more than 0.5 Å.

The term “positively charged amino acid” includes any naturally occurring or unnatural amino acid having a positively charged side chain under normal physiological conditions. Examples of positively charged naturally occurring amino acids are arginine, lysine and histidine.

By “primer” is meant an oligonucleotide which, when paired with a strand of DNA, is capable of initiating the synthesis of a primer extension product in the presence of a suitable polymerising agent. The primer is preferably single-stranded for maximum efficiency in amplification but may alternatively be double-stranded. A primer must be sufficiently long to prime the synthesis of extension products in the presence of the polymerisation agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotides, although it may contain fewer nucleotides. Primers can be large polynucleotides, such as from about 200 nucleotides to several kilobases or more. Primers may be selected to be “substantially complementary” to the sequence on the template to which it is designed to hybridise and serve as a site for the initiation of synthesis. By “substantially complementary”, it is meant that the primer is sufficiently complementary to hybridise with a target nucleotide sequence. Preferably, the primer contains no mismatches with the template to which it is designed to hybridise but this is not essential. For example, non-complementary nucleotides may be attached to the 5′ end of the primer, with the remainder of the primer sequence being complementary to the template. Alternatively, non-complementary nucleotides or a stretch of non-complementary nucleotides can be interspersed into a primer, provided that the primer sequence has sufficient complementarity with the sequence of the template to hybridise therewith and thereby form a template for synthesis of the extension product of the primer.

The term “root mean square deviation” means the square root of the arithmetic mean of the squares of the deviations from the mean. It is a way to express the deviation or variation from a trend or object. For purposes of this invention, the “root mean square deviation” defines the variation in the Cα atoms of a protein from the Cα atoms of Bcl-w or a active site portion thereof, as defined by the structure coordinates of Bcl-w described herein.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

“Structural coordinates” are the Cartesian coordinates corresponding to an atom's spatial relationship to other atoms in a molecule or molecular complex. Structural coordinates may be obtained using x-ray crystallography techniques or NMR techniques, or may be derived using molecular replacement analysis or homology modelling. Various software programs allow for the graphical representation of a set of structural coordinates to obtain a three dimensional representation of a molecule or molecular complex. The structural coordinates of the present invention may be modified from the original set provided in TABLE 1 by mathematical manipulation, such as by inversion or integer additions or subtractions. As such, it is recognised that the structural coordinates of the present invention are relative, and are in no way specifically limited by the actual x, y, z coordinates of TABLE 1.

The term “unnatural amino acids” means amino acids that are not naturally found in proteins. Examples of unnatural amino acids used herein include racemic mixtures of selenocysteine and selenomethionine. In addition, unnatural amino acids include the D or L forms of nor-leucine, para-nitrophenylalanine, homophenylalanine, para-fluorophenylalanine, 3-amino-p2-benzylpropionic acid, homoarginine, and D-phenylalanine.

By “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into a cell, is integrated into the genome of the recipient cell and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the cell into which the vector is to be introduced. The vector may also include a selection marker such as an antibiotic resistance gene that can be used for selection of suitable transformants. Examples of such resistance genes are well known to those of skill in the art.

2. Abbreviations

The following abbreviations are used throughout the application:

| | | |
|---|---|---|
| A = | Ala = | Alanine |
| V = | Val = | Valine |
| L = | Leu = | Leucine |
| I = | Ile = | Isoleucine |
| P = | Pro = | Proline |
| F = | Phe = | Phenylalanine |
| W = | Trp = | Tryptophan |
| M = | Met = | Methionine |
| G = | Gly = | Glycine |
| S = | Ser = | Serine |
| T = | Thr = | Threonine |
| C = | Cys = | Cysteine |
| Y = | Tyr = | Tyrosine |
| N = | Asn = | Asparagine |
| Q = | Gln = | Glutamine |
| D = | Asp = | Aspartic Acid |
| E = | Glu = | Glutamic Acid |
| K = | Lys = | Lysine |
| R = | Arg = | Arginine |
| H = | His = | Histidine |

3. Solution Structure

The present invention relates to the three dimensional structure of a Bcl-w (Bcl-wΔC10) derivative, and more specifically, to the solution structure of that derivative as determined using multi-dimensional NMR spectroscopy and various computer modelling techniques. The solution coordinates of this derivative (disclosed herein in TABLE 1) are useful for a number of applications, including, but not limited to, the characterisation of a three dimensional structure of Bcl-w and its variants or derivatives, as well as the visualisation, identification and characterisation of Bcl-w active sites, including the site of BH3 ligand binding to Bcl-w. The active site structures may then be used to predict the orientation and binding affinity of a designed or selected agonist or antagonist of Bcl-w, of a Bcl-w variant, derivative or analogue, or of a complex comprising Bcl-w or variant or derivative thereof. Accordingly, the invention is particularly directed to the three dimensional structure of a Bcl-w active site including, but not limited to, the BH3 ligand binding site.

The Bcl-w, Bcl-w variant, derivative or analogue, or complex in solution suitably comprises amino acid residues 43-150 of Bcl-w, more suitably amino acid residues 43-173 of Bcl-w, preferably amino acid residues 43-173 as set forth in SEQ ID NO: 1, more preferably amino acid residues 43-173 as set forth in SEQ ID NO: 2 and still more preferably amino acid residues 1-183 as set forth in SEQ ID NO: 2, or conservative substitutions thereof. In an especially preferred embodiment, the solution contains a polypeptide comprising the sequence set forth in SEQ ID NO:2, which defines a Bcl-w derivative that lacks the last 10 amino acid residues of Bcl-w and that has Ala 182 substituted with a glutamate residue or modified form thereof (referred to herein as Bcl-wΔC10).

Preferably, the Bcl-w or Bcl-w variant, derivative or analogue, or complex in solution is either unlabelled, ¹⁵N enriched or ¹⁵N, ¹³C enriched. In addition, the secondary structure of the Bcl-w or Bcl-w variant, derivative or analogue, or complex in the solutions of the present invention suitably comprises eight α-helices. In this regard, α1 comprises amino acid residues Thr10 to Gln24 of Bcl-w, α2 comprises amino acid residues His43 to Thr55 of Bcl-w, α3 comprises amino acid residues Ser62 to Leu68 of Bcl-w, α4 comprises amino acid residues Gln76 to Phe87 of Bcl-w, α5 comprises amino acid residues Trp93 to Val 111 of Bcl-w, α6 comprises amino acid residues Glu114 to Thr132 of Bcl-w, α7 comprises amino acid residues Leu134 to Ser141 of Bcl-w, and α8 comprises amino acid residues Trp144 to Leu150 of Bcl-w. The secondary structure preferably further comprises a ninth α-helix, α9, which comprises amino acid residues Glu157 to Val173 of Bcl-w and which forms part of the C-terminal region of Bcl-w. Preferably, the secondary structure further comprises amino acid residues Leu174 to Leu183, which forms another part of the C-terminal region.

The Bcl-w or Bcl-w variant, derivative or analogue, or complex in solution is suitably analysed by NMR techniques as known in the art, including standard 2D, 3D and 4D triple resonance NMR techniques, to generate NMR spectra. Typically, these spectra are then analysed to obtain NMR resonance assignments and structural constraint assignments, which may contain hydrogen bond, distance, dihedral angle, coupling constant, chemical shift and dipolar coupling constant constraints.

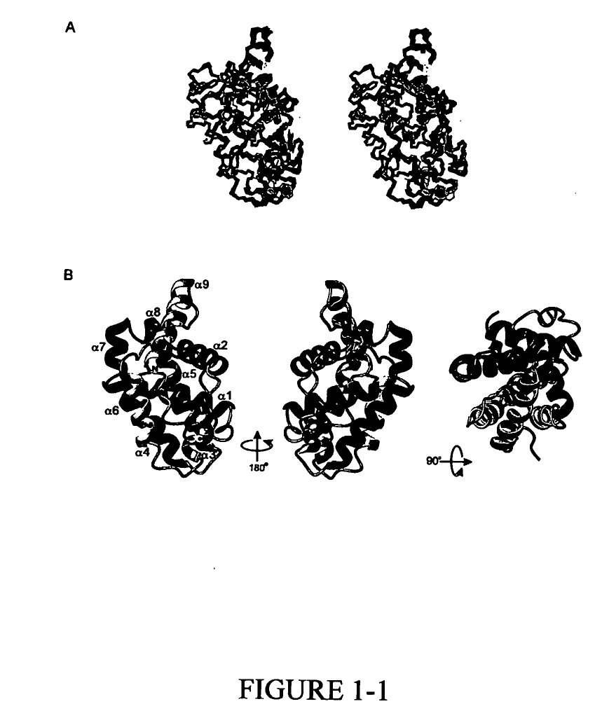

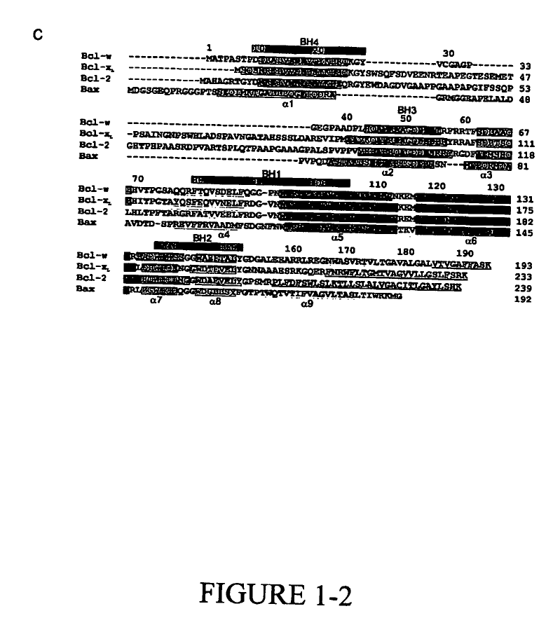

In accordance with a non-limiting embodiment of the present invention, essentially complete, sequence-specific, backbone and side chain assignments for Bcl-wΔC10 were determined using a series of heteronuclear 3D NMR experiments (Sattler et al., 1999). Structures were calculated using a total of 3871 constraints (Table 2). FIG. 1A shows the superposition of the final 20 lowest-energy structures over the backbone atoms (N, Cα, C′) of residues 8-183. The structural statistics for the ensemble are shown in Table 2 and demonstrate that the NMR structures are both energetically reasonable and have acceptable covalent geometry. The N-terminal 13 residues, including the 5 cloning artefacts (GPLGS), lack any long-range distance constraints and are disordered in solution. In addition the amide protons for residues 59 and 114-115 are in short solvent-accessible loops that exchange rapidly with solvent and are not observable.

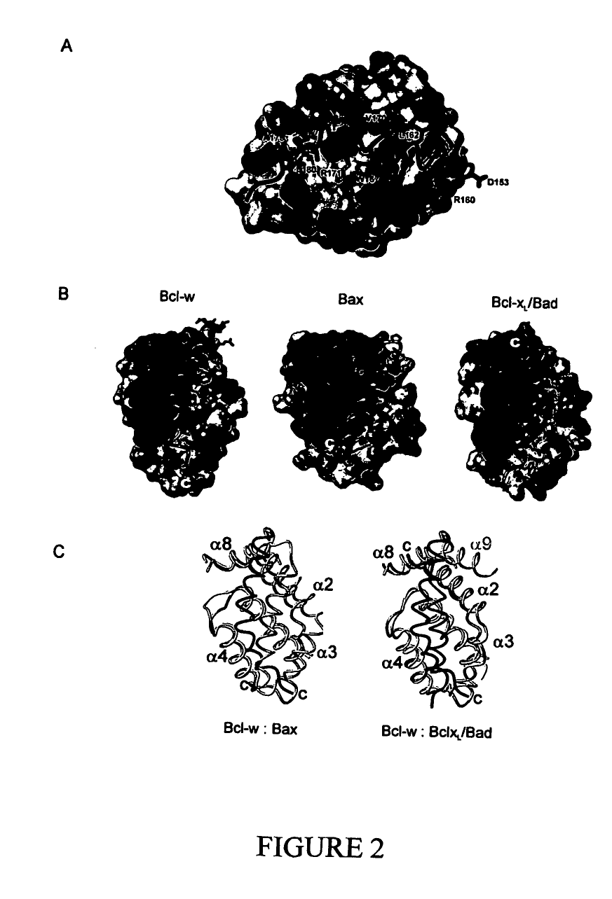

As depicted in FIG. 1, Bcl-wΔC10 is an α-helical protein containing a well-defined core formed by a central hydrophobic helix, α5, (residues 93-111) and flanking amphipathic helices α1 (residues 10-24), α2 (residues 43-56), α3 (residues 62-68), α4 (residues 76-87) and α6 (residues 116-132) (FIG. 1). The amphipathic helices pack closely onto α5 and it is therefore largely inaccessible to solvent (FIG. 1B). The helices are connected by a series of well-defined loops. The α1-α2 loop is 13 residues in length and has an extended conformation with a turn in the centre that packs onto α1. Although the ends of this loop have few contacts, the central region has a number of specific contacts and is ordered (FIG. 1A). Short ordered loops connect the remaining helices although some local disorder is seen for the α5-α6 loop, reflecting the fact that the assignments in this region are not complete. Helix α7 (residues 134-141) is essentially continuous with α6 except for a sharp bend, indicated by a change in the coupling constants, which occurs at residue 133 and disrupts the two helices. At the base of α7 lies helix α8 (residues 144-150) that primarily contacts α2. A sharp turn containing two glycine residues connects α8 to helix α9 (residues 157-173). As a consequence of this turn α9 is folded back onto the structure and the C-terminus of α9 makes contacts with residues at the N-terminus of α5 and the α4-α5 loop (FIG. 2A). Following α9 is a region (residues 174-183) of extended but ordered structure. This extended region lies in a groove that is principally formed by residues located in α3, α4 and the N-terminus of α5. The position of the extended region is stabilised by a series of hydrophobic interactions between the tail and residues in α3-α5.

The presence of the C-terminus in the hydrophobic groove means that Bcl-wΔC10 is a compact globular molecule with no significant hydrophobic surface attributes. The most distinct surface feature of Bcl-w is a region of negative electrostatic potential formed by residues from α1, α1-α2 loop, α5-α6 loop, α6 and α7. A smaller region of positive potential, which is largely formed by basic residues in α9, is seen on the opposite face of the molecule.

A binding site for BH3 ligands is provided by the hydrophobic groove bounded by residues on helices α2-α5 and α8. The binding site is formed from residues on the BH1, BH2 and BH3 domains of Bcl-w (FIG. 1C) that are brought into close spatial proximity by the three-dimensional fold of the molecule. The C-terminal helix (α9, residues 157-173) and extended region (residues 174-183) of Bcl-wΔC10 lie over the surface formed from the BH1, BH2 and BH3 domains of Bcl-w that bind BH3-ligands as defined by the structures of the complexes of C-terminally truncated Bcl-x_{L} bound to BH3 peptides of Bak (Sattler et al., 1997) and Bad- (Petros et al., 2000). In addition to the Bcl-x_{L} complex structures there is a structure of a pro-apoptotic molecule, Bax, (Suzuki et al., 2000), which is similar to that of Bcl-w, in that the C-terminal helix of Bax lies over the surface created by the BH1, BH2 and BH3 domains of Bax (FIG. 2). The C-terminal residues of Bcl-w envelop residues located in α2, α2-α3 loop, α3, α3-α4 loop, α4, α4-α5 loop, α5 and α8 and covers approximately 1100 Å² (as judged by the solvent accessibility calculated in the program MOLMOL (Koradi et al., 1996) (see TABLE 3, which lists the changes). These data suggest that the C-terminal tail of Bcl-wCΔ10 occludes the BH3 binding site.

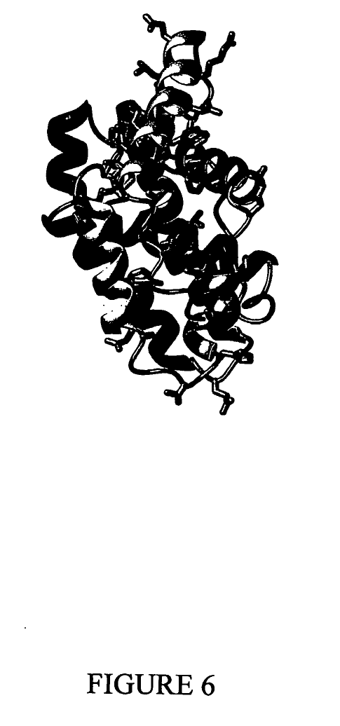

A ¹⁵N-NOESY-HSQC spectrum of Bcl-wΔC10 complexed with Bim-BH3 peptide (Sequence: DLRPEIRIAQELRRIGDEFNETYTRR; residues 53-78 of murine Bim_{L} SwissProt accession number 054918) showed that amide resonances from residues 49-51, 53 (on α2); 58, 61, (α2-α3 loop); 63, 64, 66, (α3); 69, 71, 75 (α3-α4 loop); 79, 83, (α4); 88, 92, (α4-α5 loop); 93, 94, 101-103, (α5); 114 (α5-α6 loop); 120, 121, 123, (α6); 134, 135, (α7); 144, 147, 148, 149, (α8); 151, (α8-α9 loop); 158-160, 162, 163, 166-170, 172, 173, (α9) 174-179, (extension) of Bcl-wΔC10 moved when compared to the unligated protein. These residues map to a face of the molecule that is consistent with the binding of peptide in the groove according to the structures determined by Fesik and co-workers for Bcl-x_{L} (Petros et al., 2000; Sattler et al., 1997), Included in the resonance changes are the C-terminal residues (see FIG. 6). The observed changes in the spectra reflect residues that either move location and/or are directly involved in binding ligand. The majority of resonances are unchanged in their chemical shifts and this indicates the lack of change to overall 3D structure of Bcl-w on binding ligand.

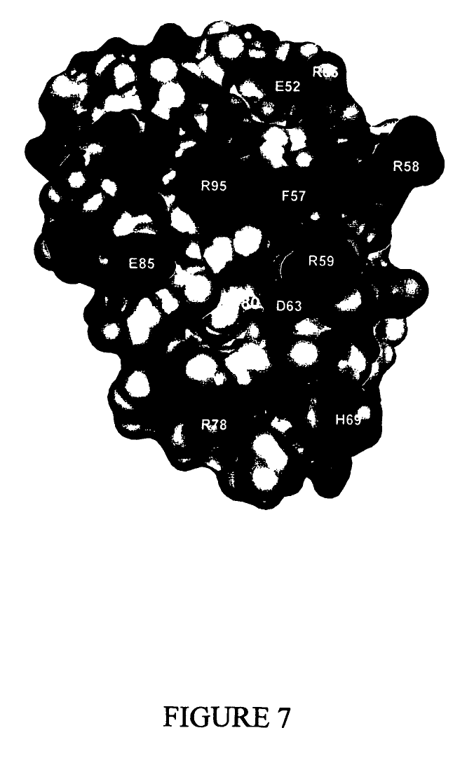

From the foregoing, there are several residues in the active site or groove of Bcl-w that can be targeted for drug design. For example, since BH3 peptides are amphipathic, (Huang and Strasser, 2000) with the hydrophobic face buried in the groove and the charged surface exposed (Petros et al., 2000; Sattler et al., 1997), it is proposed that these hydrophobic residues contact with residues in the base of the groove. The side chain of L180, in the C-terminus of Bcl-w, is buried in a pocket created by residues Arg59, Asp63, Leu64, Gln67, Phe79, Val82, Phe102, and Leu106 (see FIG. 7). Accordingly, a binding pocket defined by the structural coordinates of those residues as set forth in TABLE 1, or a binding pocket whose root mean square deviation from the structure coordinates of the Cα atoms of those residues of not more than 1.1 Å, is considered to define at least a portion of the active site of the invention and provides inter alia a target for the design of BH3-like ligands of Bcl-w.

In another example, the residues that are occluded by the C-terminal region of Bcl-w may be suitable targets as they provide many of the residues that are directly involved in binding, according to structural studies on C-terminally truncated Bcl-x_{L} and its complexes with Bak and Bad (Petros et al., 2000; Sattler et al., 1997) and the present studies on the complex. These include, but are not limited to, residues that are within 5 Å of residues in the C-terminal helix and tail (residues 153-183) of Bcl-wΔC10 such as Gln44, Ala45, Ala48, Ala49, Gly50, Glu52, Phe53, Arg56, Phe57, Arg58, Arg59, Asp63, Leu64, Ala66, Gln67, His69, Val70, Arg78, Phe79, Gln81, Val82, Ser83, Glu85, Leu86, Phe87, Gln88, Gly89, Gly90, Pro91, Asn92, Trp93, Gly94, Arg95, Val97, Phe99, Phe102, Leu106, Phe147, Thr148, Ala149, Leu150, Tyr151 and Gly152, and residues that are within 8 Å of residues in the C-terminal helix and tail of Bcl-wΔC10 such as Met46, Arg47, Asp51, Glu54, Thr55, Thr60, Ser62, Ala65, Leu68, Thr71, Ala75, Gln76, Gln77, Thr80, Asp84, Leu96, Ala98, Gly103, Trp137, Ser141 and Glu146. The residues, therefore, are proposed to define another part of the Bcl-w active site. Accordingly, a surface defined by the structural coordinates of at least three of those residues as set forth in TABLE 1, or a surface whose root mean square deviation from the structure coordinates of the Cαatoms of those residues of not more than 1.1 Å, is considered to define at least another portion of the active site of the invention.

The present inventors consider that charged residues play a role in the binding of BH3 ligands to their target and that consideration of the charge and shape complementarity is desirable, therefore, in the design of antagonists of Bcl-w pro-survival activity. In this connection, there are conserved charged amino acid residues in the sequences of Bcl-w, Bcl-x_{L}, Bcl-2 that line, or are in close proximity to, the binding groove that can be targeted in molecular design. For instance, suitable charged residues of this type include, but are not limited to, Glu52, Arg56, Arg58, Asp63, Glu85, Arg95, Lys113, in Bcl-w (see FIGS. 1 and 7). In particular, the highly conserved Arg95 of Bcl-w becomes exposed on removal of the C-terminal tail and in the published structures of complexes (Petros et al., 2000; Sattler et al., 1997) the equivalent residue in BCl-x_{L} (Arg139) is in close proximity to the conserved aspartate of the BH3 ligand (equivalent to residue Asp99 on Bim_{L}). Mutation of Arg95 to Ala abrogates the binding of Bim to Bcl-w indicating its importance in the Bcl-w binding pocket. Accordingly, this charged residue represents an attractive target for drug design.

Further more, the mutation F57R in Bcl-w abrogates its pro-survival activity while the mutation F57A has little effect on Bcl-w pro-survival activity. This Phe residue sits in a cluster of basic residues including Arg56, Arg58 and Arg59 (FIG. 7) and provides another possible target for a BH3 mimetic.

It is also proposed that because the Bcl-w basic amino acid residue Lys113 is conserved in Bcl-2 and Bcl-x_{L} and is within 12 Å of the C-terminal helix and tail (residues 153 to 183) of Bcl-wΔC10, it plays a role as a counter ion to the C-terminal residues. This residue represents another possible target for drug design.

Those of skill in the art will appreciate that a set of structure coordinates for a protein or a protein-complex or a portion thereof, is a relative set of points that define a shape in three dimensions. Thus, it is possible that an entirely different set of coordinates could define a similar or identical shape. Moreover, slight variations in the individual coordinates will have little effect on overall shape. In terms of active sites, these variations would not be expected to significantly alter the nature of ligands that could associate with those sites. These variations in coordinates may be generated because of mathematical manipulations of the Bcl-wΔC10 structure coordinates. For example, the structure coordinates set forth in TABLE 1 could be manipulated by fractionalisation of the structure coordinates, integer additions or subtractions to sets of the structure coordinates, inversion of the structure coordinates or any combination of the above.

Alternatively, modifications in the solution structure due to mutations, additions, substitutions, and/or deletions of amino acids, or other changes in any of the components of the solution that is the subject of the NMR could also account for variations in structure coordinates. If such variations are within an acceptable standard error as compared to the original coordinates, the resulting three-dimensional shape is considered to be the same. Thus, for example, a ligand that bound to the active site of Bcl-wΔC10 would also be expected to bind to another site or binding pocket whose structure coordinates defined a shape that fell within the acceptable error. Accordingly, an active site defined by the structural coordinates of the amino acid residues defined above; or an active site whose root mean square deviation from the structure coordinates of the Cα atoms of those residues of not more than about 1.1 Å is considered a Bcl-w-like active site of this invention.

It will be readily apparent to the skilled artisan that the numbering of amino acid residues in variants or other isoforms of Bcl-w may be different than that set forth in TABLE A. Corresponding amino acid residues in such variants or isoforms are easily identified by visual inspection of the amino acid sequences or by using commercially available homology software programs, as for example described herein.

Various computational analyses may be used to determine whether a protein or the active site portion thereof is sufficiently similar to the Bcl-w active site described above. Such analyses may be carried out in well known software applications, such as the Molecular Similarity application of SYBYL (Tripos, Inc., 1699 South Hanley Rd., St. Louis, Mo. 63144, USA) or QUANTA (Molecular Simulations Inc., San Diego, Calif., version 4.1), and as described in the accompanying User's Guides.

4. Uses of the Structure Coordinates

4.1 Computer System Related Embodiments

Molecular modelling methods known in the art may be used to identify an active site or binding pocket of Bcl-w, a Bcl-w complex, or of a Bcl-w variant or derivative or analogue. Specifically, the solution structural coordinates provided by the present invention may be used to characterise a three dimensional structure of the Bcl-w molecule, molecular complex or Bcl-w variant or derivative or analogue. From such a structure, putative active sites may be computationally visualised, identified and characterised based on the surface structure of the molecule, surface charge, steric arrangement, the presence of reactive amino acid residues, regions of hydrophobicity or hydrophilicity, etc. Such putative active sites may be further refined using chemical shift perturbations of spectra generated from various and distinct Bcl-w complexes, competitive and non-competitive inhibition experiments, and/or by the generation and characterisation of Bcl-w or ligand mutants to identify critical residues or characteristics of the active site.

The identification of putative active sites of a molecule or molecular complex is of great importance, as most often the biological activity of a molecule or molecular complex results from the interaction between an agent and one or more active sites of the molecule or molecular complex. Accordingly, the active sites of a molecule or molecular complex are the best targets to use in the design or selection of modulators that affect the activity of the molecule or molecular complex.

The present invention is directed to an active site of Bcl-w, a Bcl-w complex or of a Bcl-w variant, derivative or analogue, that, as a result of its shape, reactivity, charge potential, etc., favourably interacts or associates with another agent (including, without limitation, a protein, polypeptide, peptide, nucleic acid, including DNA or RNA, molecule, compound, antibiotic or drug). Preferably, the present invention is directed to an active site of a BH3 ligand-binding protein or peptide, as broadly described above.

In order to use the structural coordinates generated for a solution structure of the present invention as set forth in TABLE 1, it is often necessary to display the relevant coordinates as, or convert them to, a three dimensional shape or graphical representation, or to otherwise manipulate them. For example, a three dimensional representation of the structural coordinates is often used in rational drug design, molecular replacement analysis, homology modelling, and mutation analysis. This is typically accomplished using any of a wide variety of commercially available software programs capable of generating three dimensional graphical representations of molecules or portions thereof from a set of structural coordinates. Such commercially available software programs are known in the art, several examples of which are listed in Section 4.2 infra.

The ready use of the subject coordinate data for molecular modelling preferably, but not essentially, requires that they be stored in a format that is useable by a computer system adapted to generate, on the basis of those data, a three-dimensional graphical representation of at least a portion of Bcl-w or structurally similar variant. Thus, in accordance with the present invention, data representing the structure coordinates of Bcl-w amino acid residues or structural coordinates having a root mean square deviation from the Cα atoms of those residues of not more than 1.1 Å and which are capable of being displayed as the three dimensional structure of at least a portion of Bcl-w or structurally similar variant thereof may be stored in a data store or database for use as part of a computer system. The database may have stored therein the entire set of structure coordinates which define the entire Bcl-wΔC10 or structurally similar variant thereof, including Bcl-w, Bcl-wΔC5 and related polypeptides, or may comprise a subset of such coordinates defining a portion of Bcl-w including, for example, its active site as defined herein.

The three-dimensional representation or structure of at least a portion of a polypeptide of interest (e.g., Bcl-w or its structurally similar variant) is understood to mean a portion of the three-dimensional surface structure or region of that polypeptide, including charge distribution and hydrophilicity/hydrophobicity characteristics, formed by at least three, more preferably at least three to ten, and even more preferably at least ten contiguous amino acid residues of the polypeptide. The contiguous residues forming such a portion may be residues which form a contiguous portion of the primary structure of the polypeptide or residues which form a contiguous portion of the three-dimensional surface of the polypeptide. Thus, the residues forming a portion of the three-dimensional structure of the polypeptide need not be contiguous in the primary sequence of the polypeptide but, rather, must form a contiguous portion of the polypeptide's surface. In a preferred embodiment, a portion of Bcl-w comprises or defines at least one Bcl-w active site binding pocket, as described herein.

Suitably, the computer system comprises a processing means for processing the data in the database to generate a molecular model having a three-dimensional shape representative of at least a portion of Bcl-w or structurally similar variant thereof. In a preferred embodiment, the processor is capable of producing a molecular model having, in addition to the three-dimensional shape, a solvent accessible surface representative of at least a portion of Bcl-w or structurally similar variant thereof.

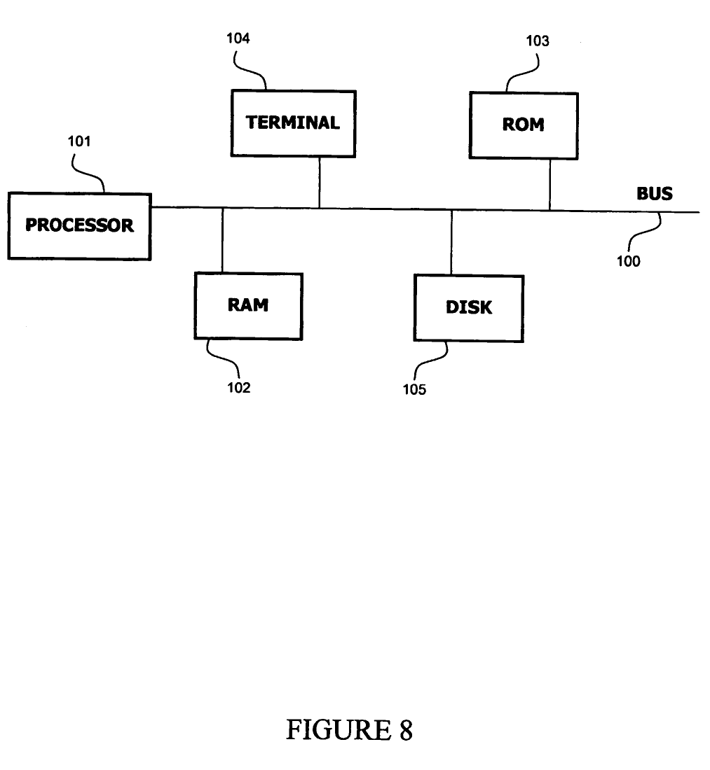

Any general or special purpose computer system is contemplated by the present invention and includes a processor in electrical communication with both a memory and at least one input/output device, such as a terminal. Such a system may include, but is not limited to, personal computers, workstations or mainframes. The processor may be a general purpose processor or microprocessor or a specialised processor executing programs located in RAM memory. The programs may be placed in RAM from a storage device, such as a disk or pre-programmed ROM memory. The RAM memory in one embodiment is used both for data storage and program execution. The computer system also embraces systems where the processor and memory reside in different physical entities but which are in electrical communication by means of a network. For example, a computer system having the overall characteristics set forth in FIG. 8 may be useful in the practice of the instant invention. More specifically, FIG. 8 is a schematic representation of a typical computer work station having in electrical communication (100) with one another via, for example, an internal bus or external network, a processor (101), a RAM (102), a ROM (103), a terminal (104), and optionally an external storage device, for example, a diskette, CD ROM, or magnetic tape (105).

In the practice of the present invention, the processing means executes a modelling program which accesses from the database data representative of the structure coordinates of at least a portion of Bcl-w or structurally similar variant thereof, to thereby construct a three-dimensional model of that molecule. Suitably, the processing means can also execute another program, a solvent accessible surface program, which uses for example the three-dimensional model of Bcl-wΔC10 or variant thereof to construct a solvent accessible surface of at least a portion of that molecule and optionally determine the solvent accessible areas of atoms. In one embodiment the solvent accessible surface program and the modelling program are the same program. In another embodiment, the modelling program and the solvent accessible surface program are different programs. In such an embodiment the modelling program may either store the three-dimensional model in a region of memory accessible both to it and to the solvent accessible surface program, or the three-dimensional model may be written to external storage, such as a disk, CD ROM, or magnetic tape for later access by the solvent accessible surface program.

As mentioned above, the Bcl-wΔC10 structural coordinate data is useful for screening and identifying chemical entities that antagonise Bcl-w. For example, the structure encoded by the data may be computationally evaluated for its ability to associate with putative ligands. Such compounds that associate with Bcl-w may antagonise Bcl-w, and are potential drug candidates. Additionally or alternatively, the structure encoded by the data may be displayed in a graphical three-dimensional representation on a computer screen. This allows visual inspection of the structure, as well as visual inspection of the structure's association with the compounds.

Thus, the present invention also encompasses an analysis method, executable by a computer system, for evaluating the potential of a compound to associate with a molecule or molecular complex comprising an active site defined by the structure coordinates of Bcl-w amino acid residues forming an active site of Bcl-w, or a variant of the molecule or molecular complex, wherein the variant comprises an active site that has a root mean square deviation from the Cα atoms of those residues of not more than about 1.1 Å. The method comprises the steps of: (a) generating a model of the active site using structure coordinates wherein the root mean square deviation between the structure coordinates and the structure coordinates of the Bcl-w amino acid residues defining a Bcl-w active site of the invention is not more than about 1.1 Å; (b) performing a fitting operation between the chemical entity and the model of the active site; and (c) quantifying the association between the chemical entity and the active site model, based on the output of said fitting operation.

The root mean square deviation is preferably determined by further using the structure coordinates of Bcl-w amino acid residues additional to those defining the Bcl-w active site. These additional amino acid residues are preferably no more than 40 Å, more preferably no more than 20 Å, even more preferably no more than 10 Å, and still more preferably no more than 8 Å from the nearest atom forming part of the Bcl-w active site of the invention. More preferably, the root mean square deviation is determined by using the structure coordinates of the all Bcl-w amino acid residues as set forth in TABLE 1.

The present invention also facilitates an analysis method, executable by a computer system, for comparing the ability of a chemical entity to associate with a first molecule or molecular complex comprising a first active site relative and the ability of that chemical entity to associate with a second molecule or molecular complex comprising a second active site. For example, this method has utility in identifying, selecting, or designing chemical entities, including antagonist compounds, that more favourably, or strongly, associate with Bcl-w than with other pro-survival Bcl-2 family members. The method suitably comprises the steps of: (a) generating a model of the first active site using structure coordinates wherein the root mean square deviation between those structure coordinates and the structure coordinates of the Bcl-w amino acid residues defining a Bcl-w active site of the invention is not more than about 1.1 Å; (b) performing a first fitting operation between the chemical entity and the model of the first active site; (c) quantifying the association between the chemical entity and the first active site model, based on the output of the first fitting operation; (d) performing a second fitting operation between the chemical entity and a model of the second active site; (e) quantifying the association between the chemical entity and the second active site model, based on the output of the second fitting operation; and (f) comparing the respective associations of the chemical entity with the first active site model and with the second active site model. From this comparison step, it is possible to determine whether the chemical entity associates more favourably with the first molecule or molecular complex than with the second molecule or molecular complex. This method is useful for identifying ligands that are selective for Bcl-w or closely related variants.

In a preferred embodiment, the second molecule or molecular complex comprises Bcl-2, Bcl-x_{L}, Mcl-1 and A1, or variant thereof. In this instance, the second binding pocket model may be a solution structural model, an X-ray crystallographic model or any other structural model of Bcl-2, Bcl-x_{L}, Mcl-1 and A1, or variant thereof.

The present invention is also directed to an analysis method, executable by a computer system, for identifying a chemical entity that associates with both a first molecule or molecular complex comprising a first active site, and a second molecule or molecular complex comprising a second active site. This method comprises the steps of: (a) generating a model of the first active site using structure coordinates wherein the root mean square deviation between the structure coordinates and the structure coordinates of the Bcl-w amino acid residues defining a Bcl-w active site of the invention is not more than about 1.1 Å; (b) performing a fitting operation between the chemical entity and the model of the first active site; (c) quantifying the association between the chemical entity and the first active site model, based on the output of the first fitting operation; (d) performing a second fitting operation between the chemical entity and a model of the second active site; (e) quantifying the association between the chemical entity and the second active site model, based on the output of the second fitting operation; and (f) comparing the respective associations of the chemical entity with the first active site model and with the second active site model. From this comparison step, it is possible to determine whether the chemical entity individually associates with both the first molecule or molecular complex and the second molecule or molecular complex, which permits the identification of ligands that can bind to Bcl-w and to one or more other Bcl-2 family members.

In another embodiment, the structural coordinates of a Bcl-w active site of the invention can be utilised in a method for identifying a potential antagonist of a molecule comprising a Bcl-w-like binding pocket. This method comprises the steps of (a) using atomic coordinates of at least three Bcl-w amino acid residues defining a Bcl-w active site as defined herein ± a root mean square deviation from the Cα atoms of those residues of not more than about 1.1 Å, to generate a three-dimensional structure of a molecule comprising a Bcl-w-like active site;

(b) employing the three-dimensional structure to identify, design or select the potential antagonist; (c) synthesising or otherwise obtaining the antagonist; and (d) contacting the antagonist with the molecule to determine the ability of the potential antagonist to interact with the molecule.

4.2 Solving the Structures of Unknown Molecules and Identification, Selection and Design of Chemical Entities that Associate with Bcl-w or Variants Thereof

The structural coordinates of the present invention permit the use of various molecular design and analysis techniques in order to solve the three dimensional structures of related molecules, molecular complexes or Bcl-w variants, derivatives or analogues. More specifically, the present invention provides a method for determining the molecular structure of a molecule or molecular complex whose structure is unknown, comprising the steps of obtaining a solution of the molecule or molecular complex whose structure is unknown, and then generating NMR data from the solution of the molecule or molecular complex. The NMR data from the molecule or molecular complex whose structure is unknown is then compared to the solution structure data obtained from the Bcl-wΔC10 solutions of the present invention. Then, 2D, 3D and 4D isotope filtering, editing and triple resonance NMR techniques are used to conform the three dimensional structure determined from the Bcl-wΔC10 solution of the present invention to the NMR data from the solution molecule or molecular complex. Alternatively, molecular replacement may be used to conform the Bcl-wΔC10 solution structure of the present invention to x-ray diffraction data from crystals of the unknown molecule or molecular complex.

Molecular replacement uses a molecule having a known structure as a starting point to model the structure of an unknown crystalline sample. This technique is based on the principle that two molecules which have similar structures, orientations and positions will diffract x-rays similarly. A corresponding approach to molecular replacement is applicable to modelling an unknown solution structure using NMR technology. The NMR spectra and resulting analysis of the NMR data for two similar structures will be essentially identical for regions of the proteins that are structurally conserved, where the NMR analysis consists of obtaining the NMR resonance assignments and the structural constraint assignments, which may contain hydrogen bond, distance, dihedral angle, coupling constant, chemical shift and dipolar coupling constant constraints. The observed differences in the NMR spectra of the two structures will highlight the differences between the two structures and identify the corresponding differences in the structural constraints. The structure determination process for the unknown structure is then based on modifying the NMR constraints from the known structure to be consistent with the observed spectral differences between the NMR spectra.

Accordingly, in one non-limiting embodiment of the invention, the resonance assignments for the Bcl-wΔC10 solution provide the starting point for resonance assignments of Bcl-wΔC10 in a new Bcl-wΔC10:“unsolved agent” complex. Chemical shift perturbations in two dimensional ¹⁵N/¹H spectra can be observed and compared between the Bcl-wΔC10 solution and the new Bcl-wΔC10:agent complex. In this way, the affected residues may be correlated with the three dimensional structure of Bcl-wΔC10 as provided by the relevant structural coordinates of TABLE 1. This effectively identifies the region of the Bcl-wΔC10:agent complex that has incurred a structural change relative to the native Bcl-wΔC10 structure. The ¹H, ¹⁵N, ¹³C and ¹³Co NMR resonance assignments corresponding to both the sequential backbone and side chain amino acid assignments of Bcl-wΔC10 may then be obtained and the three dimensional structure of the new Bcl-wΔC10:agent complex may be generated using standard 2D, 3D and 4D triple resonance NMR techniques and NMR assignment methodology, using the Bcl-wΔC10 solution structure, resonance assignments and structural constraints as a reference. Various computer fitting analyses of the new agent with the three dimensional model of Bcl-wΔC10 may be performed in order to generate an initial three dimensional model of the new agent complexed with Bcl-wΔC10, and the resulting three dimensional model may be refined using standard experimental constraints and energy minimisation techniques in order to position and orient the new agent in association with the three dimensional structure of Bcl-wΔC10. An especially preferred embodiment of this type is described in Section 3 supra in relation to the ¹⁵N-NOESY-HSQC spectrum of Bcl-wΔC10 complexed with Bim-BH3 peptide.

The present invention further provides that the structural coordinates of the present invention may be used with standard homology modelling techniques in order to determine the unknown three-dimensional structure of a molecule or molecular complex. Homology modelling involves constructing a model of an unknown structure using structural coordinates of one or more related protein molecules, molecular complexes or parts thereof (i.e., active sites). Homology modelling may be conducted by fitting common or homologous portions of the protein whose three dimensional structure is to be solved to the three dimensional structure of homologous structural elements in the known molecule, specifically using the relevant (i.e., homologous) structural coordinates provided by TABLE 1 herein. Homology may be determined using amino acid sequence identity, homologous secondary structure elements, and/or homologous tertiary folds. Homology modelling can include rebuilding part or all of a three dimensional structure with replacement of amino acid residues (or other components) by those of the related structure to be solved.

Accordingly, a three dimensional structure for the unknown molecule or molecular complex may be generated using the three dimensional structure of the Bcl-wΔC10 molecule of the present invention, refined using a number of techniques well known in the art, and then used in the same fashion as the structural coordinates of the present invention, for instance, in applications involving molecular replacement analysis, homology modelling, and rational drug design.

Determination of the three dimensional structure of Bcl-wΔC10, its BH3 ligand binding active site, and other binding sites, is critical to the rational identification and/or design of agents that may act as antagonists of Bcl-w, such as inhibitors of BH3 ligand binding to Bcl-w. This is advantageous over conventional drug assay techniques, in which the only way to identify such an agent is to screen thousands of test compounds until an agent having the desired inhibitory effect on a target compound is identified. Necessarily, such conventional screening methods are expensive, time consuming, and do not elucidate the method of action of the identified agent on the target compound. Using such a three dimensional structure, researchers identify putative binding sites and then identify or design agents to interact with these binding sites. These agents are then screened for an inhibitory effect upon the target molecule. In this manner, not only are the number of agents to be screened for the desired activity greatly reduced, but the mechanism of action on the target compound is better understood.

Thus, in accordance with the present invention, a potential Bcl-w antagonist may now be evaluated for its ability to bind a Bcl-w-like active site prior to its actual synthesis and testing. If a proposed compound is predicted to have insufficient interaction or association with the active site, preparation and testing of the compound is obviated. However, if the computer modelling indicates a strong interaction, the compound may then be obtained and tested for its ability to bind to Bcl-w. Testing to confirm binding and/or inhibition may be performed using any suitable assay. Exemplary assays of this type are described below in the preferred embodiments. In this manner, synthesis of inoperative compounds may be avoided.

In a preferred embodiment, the potential Bcl-w antagonist may also be evaluated for its ability to bind an active site of another Bcl-2 pro-survival family member or variant thereof. In one embodiment, the computer modelling preferably indicates a weak interaction between the potential Bcl-w antagonist and the other Bcl-2 pro-survival family member or variant thereof. In another embodiment, the computer modelling preferably indicates a strong interaction between the potential Bcl-w antagonist and the other Bcl-2 pro-survival family member or variant thereof. This interaction may be assayed using suitable receptor binding assays for the other Bcl-2 pro-survival family member or variant thereof, as for example described below in the preferred embodiments.

The design of chemical entities that associate with or antagonise Bcl-w generally involves consideration of two factors. First, the compound must be capable of physically and structurally associating with Bcl-w. Non-covalent molecular interactions important in the association of Bcl-w with its substrate include hydrogen bonding, van der Waals and hydrophobic interactions. Second, the compound must be able to assume a conformation that allows it to associate with Bcl-w. Although certain portions of the compound will not directly participate in this association with Bcl-w, those portions may still influence the overall conformation of the molecule. This, in turn, may have a significant impact on potency. Such conformational requirements include the overall three-dimensional structure and orientation of the chemical entity or compound in relation to all or a portion of the active site, or the spacing between functional groups of a compound comprising several chemical entities that directly interact with Bcl-w.

A potential antagonist of a Bcl-w-like active site may be computationally evaluated by means of a series of steps in which chemical entities or fragments are screened and selected for their ability to associate with the Bcl-w-like active site. One skilled in the art may use one of several methods to screen chemical entities or fragments for their ability to associate with a Bcl-w-like binding pocket. This process may begin by visual inspection of, for example, a Bcl-w-like binding pocket on the computer screen based on the Bcl-wΔC10 structure coordinates in TABLE 1 or other coordinates which define a similar shape generated from the database.

Selected fragments or chemical entities may then be positioned in a variety of orientations, or docked, within that binding pocket as defined above. Docking may be accomplished using software such as SYBYL and QUANTA, followed by energy minimisation and molecular dynamics with standard molecular mechanics force fields, such as CHARMM and AMBER.

Specialised computer programs may also assist in the process of selecting fragments or chemical entities. These include but are not limited to:
GRID (Goodford, 1985, J. Med. Chem. 28: 849-857). GRID is available from Oxford University, Oxford, UK.
MCSS (Miranker et al., 1991, Proteins: Structure, Function and Genetics 11: 29-34). MCSS is available from Molecular Simulations, San Diego, Calif., USA.
AUTODOCK (Goodsell et al., 1990, Proteins: Structure, Function, and Genetics 8: 195202). AUTODOCK is available from Scripps Research Institute, La Jolla, Calif., USA.
DOCK (Kuntz et al., 1982, J. Mol. Biol. 161: 269-288). DOCK is available from University of California, San Francisco, Calif., USA.
UNITY a 3D database searching program available from Tripos Inc., St. Louis, Mo., USA.

Once suitable chemical entities or fragments have been selected, they can be designed or assembled into a single compound or complex. Assembly may be preceded by visual inspection of the relationship of the fragments to each other on the three-dimensional image displayed on a computer screen in relation to the structure coordinates of Bcl-wΔC10. This would be followed by manual model building using software such as SYBYL or QUANTA.

Useful programs to aid one of skill in the art in connecting the individual chemical entities or fragments include but are not restricted to:
CAVEAT (Bartlett et al., 1989, Special Pub., Royal Chem. Soc. 78: 182-196; Lauri and Bartlett, 1994, J. Comput. Aided Mol. Des. 8: 51-66). CAVEAT is available from the university of California, Berkeley, Calif., USA.
3D Database systems such as ISIS (MDL Information Systems, San Leandro, Calif., USA). This area is reviewed in Y. C. Martin (1992, J. Med. Chem. 35: 2145-2154).
HOOK (Eisen et al., 1994, Proteins: Struct., Funct., Genet. 19: 199-221). HOOK is available from Molecular Simulations, San Diego, Calif., USA.

Instead of proceeding to build an antagonist of a Bcl-w-like binding pocket in a step-wise fashion one fragment or chemical entity at a time as described above, antagonistic or other Bcl-w-binding compounds may be designed as a whole or “de novo” using either an empty binding site or optionally including some portion(s) of a known inhibitor(s). There are many de novo ligand design methods including but not limited to:
LUDI (H. -J. Bohm, 1992, J. Comp. Aid. Molec. Design 6: 61-78). LUDI is available from Molecular Simulations Incorporated, San Diego, Calif., USA.
LEGEND (Nishibata et al., 1991, Tetrahedron 47: 8985). LEGEND is available from Molecular Simulations Incorporated, San Diego, Calif., USA.
LeapFrog (available from Tripos Inc., St. Louis, Mo., USA).
SPROUT (Gillet et al., 1993, J. Comput. Aided Mol. Design 7: 127-153). SPROUT is available from the University of Leeds, UK.

Other molecular modelling techniques may also be employed in accordance with this invention (see, e.g., Cohen et al., 1990, J. Med. Chem. 33: 883-894; see also, Navia and Murcko, 1992, Current Opinions in Structural Biology 2: 202-210; Balbes et al., “A Perspective of Modern Methods in Computer-Aided Drug Design”, in Reviews in Computational Chemistry, Vol. 5, K. B. Lipkowitz and D. B. Boyd, Eds., VCH, New York, pp. 337-380 (1994); see also, Guida, 1994, Curr. Opin. Struct. Biology 4: 777-781).

Once a compound has been designed or selected by the above methods, the efficiency with which that entity may bind to a Bcl-w active site may be tested and optimised by computational evaluation. For example, an effective Bcl-w active site antagonist must preferably demonstrate a relatively small difference in energy between its bound and free states (i.e., a small deformation energy of binding). Thus, the most efficient Bcl-w active site antagonists should preferably be designed with a deformation energy of binding of not greater than about 10 kcal/mole, more preferably, not greater than 7 kcal/mole. Bcl-w active site antagonists may interact with the binding pocket in more than one of multiple conformations that are similar in overall binding energy. In those cases, the deformation energy of binding is taken to be the difference between the energy of the free entity and the average energy of the conformations observed when the antagonist binds to the protein.

An entity designed or selected as binding to a Bcl-w-like binding pocket may be further computationally optimised so that in its bound state it would preferably lack repulsive electrostatic interaction with the target protein and with the surrounding water molecules. Such non-complementary electrostatic interactions include repulsive charge-charge, dipole-dipole and charge-dipole interactions. A designed or selected chemical entity may be further computationally optimised so that it has sufficient lipophilicity to penetrate the blood brain barrier. Using these modelling and optimisation techniques, it will be possible to design Bcl-w active site antagonists with tight binding capacity and capable of displacing the C-terminal tail of Bcl-w to enable entry and binding into that site.

Specific computer software is available in the art to evaluate compound deformation energy and electrostatic interactions. Examples of programs designed for such uses include but are not limited to:
Gaussian 94, revision C (M. J. Frisch, Gaussian, Inc., Pittsburgh, Pa., USA, 1995); AMBER, version 4.1 (P. A. Kollman, University of California at San Francisco, USA, 1995).
QUANTA/CHARMM (Molecular Simulations, Inc., San Diego, Calif., USA, 1995).
INSIGHT II/DISCOVER (Molecular Simulations, Inc., San Diego, Calif., USA, 1995).
DelPhi (Molecular Simulations, Inc., San Diego, Calif., USA, 1995).
AMSOL (Quantum Chemistry Program Exchange, Indiana University, USA).

These programs may be implemented, for instance, using a Silicon Graphics workstation such as an IRIS 4D/35 or an Indigo2 with “IMPACT” graphics. Other hardware systems and software packages will be known to those skilled in the art.

Another approach enabled by this invention, is the computational screening of small molecule databases for chemical entities or compounds that can bind in whole, or in part, to a Bcl-w active site. In this screening, the quality of fit of such entities to the binding site may be judged either by shape complementarity or by estimated interaction energy (Meng et al., 1992, J. Comp. Chem. 13: 505-524).

According to another aspect, the invention provides compounds, which associate with a Bcl-w-like active site, produced or identified by the method as set forth above.

Once a Bcl-w-binding compound has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analysed for efficiency of fit to Bcl-w by the same computer methods described above.

5. Bcl-w Variants

The present invention also enables the production of variants of Bcl-w and the solving of their structures. More particularly, by virtue of the present invention, the location of the active site permits the identification of desirable sites for structural alteration, which includes substitution, addition or deletion of at least one amino acid residue. Such an alteration may be directed to a particular site or combination of sites of wild-type Bcl-w may be chosen for alteration. Similarly, a location on, at or near the protein surface may be replaced, resulting in an altered surface charge of one or more charge units, as compared to the wild-type protein. Alternatively, an amino acid residue in Bcl-w may be chosen for replacement based on its hydrophilic or hydrophobic characteristics.

Such variants may be characterised by any one of several different properties as compared with wild-type Bcl-w. For example, such variants may have altered surface charge of one or more charge units, or have increased stability, or altered ligand specificity, or altered specific activity, in comparison with wild-type Bcl-w.

The variants of Bcl-w may be prepared in a number of ways. For example, the wild-type sequence of Bcl-w may be altered in those sites identified using this invention as desirable for alteration, by means of oligonucleotide-directed mutagenesis or other conventional methods, e.g. deletion. Alternatively, variants of Bcl-w may be generated by the site-specific replacement of a particular amino acid with an unnaturally occurring amino acid. In addition, Bcl-w variants may be generated through replacement of an amino acid residue, or a particular cysteine or methionine residue, with selenocysteine or selenomethionine. This may be achieved by growing a host organism capable of expressing either the wild-type or variant polypeptide on a growth medium depleted of either natural cysteine or methionine (or both) but enriched in selenocysteine or selenomethionine (or both).

A parent Bcl-w or Bcl-w derivative-encoding polynucleotide can be mutated using standard mutagenesis techniques including, but not limited to, random mutagenesis (e.g., transposon mutagenesis), oligonucleotide-mediated (or site-directed) mutagenesis, PCR mutagenesis and cassette mutagenesis. The mutated polynucleotide so produced, or produced by any alternative methods known in the art, can be expressed using suitable expression systems and the variant polypeptides produced in these systems may be purified by a variety of conventional steps and strategies, including those used to purify wild-type Bcl-w. Alternatively, the recombinant polypeptides may be conveniently prepared by a person skilled in the art using standard protocols as for example described in Sambrook, et al., MOLECULAR CLONING. A LABORATORY MANUAL (Cold Spring Harbor Press, 1989), in particular Sections 16 and 17; Ausubel et al., CURRENT PROTOCOLS IN MOLECULAR BIOLOGY (John Wiley & Sons, Inc. 1994-1998), in particular Chapters 10 and 16; and Coligan et al., CURRENT PROTOCOLS IN PROTEIN SCIENCE (John Wiley & Sons, Inc. 1995-1997), in particular Chapters 1, 5 and 6.

Once the Bcl-w variants have been generated in the desired location, the variants may be tested for any one of several properties of interest.

For example, variants may be screened for an altered charge at physiological pH. This is determined by measuring the variant Bcl-w isoelectric point (pI) in comparison with that of the wild-type parent. Isoelectric points may be measured by gel-electrophoresis according to the method of Wellner (1971, Analyt. Chem. 43: 597). A variant with an altered surface charge is suitably a Bcl-w polypeptide containing a replacement amino acid located at the surface of the enzyme, as provided by the structural information of this invention, and an altered pI.

6. Pharmaceutical Compositions

Agonist or antagonist compounds identified, designed or selected based on the methods and structures of the present invention might be useful as important leads for the development of compositions to treat a Bcl-w or other pro-survival Bcl-2 family member-mediated disease or condition, including diseases or conditions associated with the activation or inactivation of apoptosis, including degenerative disorders characterised by inappropriate cell proliferation or inappropriate cell death, respectively. Disorders characterised by inappropriate cell proliferation include, for example, inflammatory conditions such as inflammation arising from acute tissue injury including, for example, acute lung injury, cancer including lymphomas, such as prostate hyperplasia, genotypic tumours, autoimmune disorders, tissue hypertrophy etc. Degenerative disorders characterised by inappropriate cell death include, for example, acquired immunodeficiency disease (AIDS), kidney disorders including polycystic kidney disease, cell death due to radiation therapy or chemotherapy, neurodegenerative diseases, such as Alzheimer's disease and Parkinson's disease, etc.

Pharmaceutical compositions of the present invention will comprise a compound identified, selected or designed using the subject three-dimensional structure (hereinafter referred to collectively as “actives”) and optionally a pharmaceutically acceptable carrier and/or diluent. Depending on the specific conditions being treated, the active(s) may be formulated and administered systemically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the actives of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation.

The actives can be formulated readily using pharmaceutically acceptable carriers well known in the art into dosages suitable for oral administration. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water.

Pharmaceutical compositions suitable for use in the present invention include compositions wherein the active ingredients are contained in an effective amount to achieve its intended purpose. The dose of active administered to a patient should be sufficient to effect a beneficial response in the patient over time such as, for example, a decrease in blood pressure. The quantity of the active(s) to be administered may depend on the patient to be treated inclusive of the age, sex, weight and general health condition thereof. In this regard, precise amounts of the active(s) for administration will depend on the judgement of the practitioner. In determining the effective amount of the active to be administered in a treatment, the practitioner may evaluate the progression of a condition to be treated or the progression of a sought-after response. In any event, those of skill in the art may readily determine suitable dosages of the active(s) of the invention.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilisers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilising processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterise different combinations of active compound doses.

Pharmaceuticals which can be used orally include push-fit capsules made of gelatin, as well as soft, sealed capsules made of gelatin and a plasticiser, such as glycerol or sorbitol. The push-fit capsules can contain the active ingredients in admixture with filler such as lactose, binders such as starches, and/or lubricants such as talc or magnesium stearate and, optionally, stabilisers. In soft capsules, the active compounds may be dissolved or suspended in suitable liquids, such as fatty oils, liquid paraffin, or liquid polyethylene glycols. In addition, stabilisers may be added.

Dosage forms of the therapeutic agents of the invention may also include injecting or implanting controlled releasing devices designed specifically for this purpose or other forms of implants modified to act additionally in this fashion. Controlled release of an agent of the invention may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes and/or microspheres.

Furthermore, one may administer the active in a targeted drug delivery system, for example, in a liposome coated with tissue-specific antibody. The liposomes will be targeted to and taken up selectively by the tissue.

The active(s) of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

For any compound used in the method of the invention, the therapeutically effective dose can be estimated initially from cell culture assays. For example, a dose can be formulated in animal models to achieve a circulating concentration range that includes the IC50 as determined in cell culture (e.g., the concentration of active(s), which achieves, for example, a half-maximal reduction in cell proliferation or cell death. Such information can be used to more accurately determine useful doses in patients.

Toxicity and therapeutic efficacy of such therapeutic agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. The data obtained from these cell culture assays and animal studies can be used in formulating a range of dosage for use in animals. The dosage of such compounds lies preferably within a range of circulating concentrations that include the ED50 with little or no toxicity. The dosage may vary within this range depending upon the dosage form employed and the route of administration utilised. The exact formulation, route of administration and dosage can be chosen by the individual practitioner in view of an animal's condition. (See for example Fingl et al., 1975, in “The Pharmacological Basis of Therapeutics”, Ch. 1 p1).

Dosage amount and interval may be adjusted individually to provide plasma levels of the active(s) which are sufficient to reduce cell death or cell proliferation. Dosage levels of between about 0.01 and about 100 mg/kg body weight per day, preferably between about 0.5 and about 75 mg/kg body weight per day of the Bcl-w agonist or antagonist compounds described herein are useful for the prevention and treatment of a Bcl-w- or other pro-survival Bcl-2 family member-mediated disease or condition. Typically, the pharmaceutical compositions of this invention will be administered from about 1 to about 5 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

The present invention will now be described with reference to the following non-limiting preferred embodiments.

### DESCRIPTION OF THE PREFERRED EMBODIMENTS

### Solution Structure of BCL-W

Initial attempts to solve the structure of Bcl-w were hampered by its poor solubility and the propensity of full-length protein to aggregate when expressed in Escherichia coli. To obtain a molecule that was amenable to study by NMR a series of truncated Bcl-w proteins was generated. The most complete sequence that was highly soluble and could be purified in large quantities was one lacking the last 10 residues (i.e., comprising 183 amino acids instead of 193 amino acids for the wild-type protein). However, acquisition of high quality spectral data from this protein was not viable due to its broad line widths. To resolve this, hydrophobic residues predicted to be solvent accessible (based on homology to Bcl-x_{L}) were mutated. One such mutation, A128E, improved the solution properties without disrupting the structure of the protein, as demonstrated by the similarity of the 2D-NOESY spectrum. Consistent with this, the A128E mutation did not affect the biological activity of full-length Bcl-w or the ability of Bcl-w to bind the BH3-only protein Bim, while deletion of the last 10 residues abolished the functional activity of Bcl-w. Longer biologically active proteins were found to have indistinguishable structures but as these samples were considerably more difficult (but not impossible) to prepare, the inventors chose to characterise Bcl-wΔC10 (A128E), referred to herein as Bcl-wΔC10.

### Structural Comparison with Other Bcl-2 Family Proteins

The overall topology of Bcl-w is very similar to that observed for other Bcl-2 family members. These include the pro-survival proteins Bcl-x_{L} (Muchmore et al., 1996), Bcl-2 (Petros et al., 2001) and the viral Bcl-2 homologue from KSHV (Huang et al., 2002) as well as the pro-apoptotic proteins Bax (Suzuki et al., 2000) and Bid (Chou et al., 1999; McDonnell et al., 1999). The position of the helices for some of the pro-survival proteins and Bax is indicated in FIG. 1C. Notably, helices α1 to α7 occupy similar positions in all structures and the hydrophobic core is conserved. When compared to the other mammalian pro-survival molecules over the core of the protein (Cα, N, C′ atoms of helices α1-α7 as defined for Bcl-w), the rmsd lies between 1.39 and 2.02 Å. In addition, the conserved BH domains present comparable surfaces in all pro-survival Bcl-2 proteins and some pro-apoptotic proteins e.g. Bax (FIG. 2B).

A number of significant differences exist. Bcl-w differs from both Bcl-2 and BCl-x_{L} in that the α1-α2 loop is both shorter and structurally well defined (FIG. 1). This 13 residue loop packs against both al and the N-terminus of α2 in Bcl-w. In contrast, the equivalent loop in Bcl-x_{L} and Bcl-2 is longer (˜58 residues) and in the structures of Bcl-x_{L}ΔC24 where the full loop is present, it is disordered as indicated by both the lack of electron density in the X-ray structure (pdb lmaz) and ¹H-¹⁵N NOE data (pdb 1l×l) (Muchmore et al., 1996). A consequence of the short well-defined α1-α2 loop in Bcl-w is that it reduces the solvent accessibility of residues 15-20 in α1 that form part of the BH4 domain. Since this domain appears essential for pro-survival activity (Borner et al., 1994; Huang et al., 1998), the α1-α2 loop might control this by modulating access to the BH4 region in Bcl-w.

The major difference between the structure of Bcl-w and those of Bcl-x_{L} and Bcl-2, is the presence and location of its C-terminal residues. The structures of Bcl-x_{L} and Bcl-2 have been determined using proteins that not only contain deletions in the α1-α2 loop but also are missing the C-terminal hydrophobic residues, hereafter these molecules are referred to as, Bcl-x_{L}ΔC24 (truncated at position 209) (Muchmore et al., 1996; Petros et al., 2000) and Bcl-2ΔC32 (truncated at position 207) (Petros et al., 2001). In addition, both proteins contained a C-terminal hexa-His tag and the residues after the last helix, α8 (FIG. 1C), are disordered. However, an additional helix (α9), that is displaced from the core of the protein and does not make any contacts with the rest of the structure, is present in KSHV-Bcl-2 (pdb 1k3k) and BCl-x_{L} complexed to BH3 peptides (pdb 1bxl and 1g5j) (Huang et al., 2002; Petros et al., 2000; Sattler et al., 1997). This suggests that in BCl-x_{L} and Bcl-2 residues beyond α8 have some helix forming ability but the location of these residues in the groove may have been destabilised by the C-terminal truncation.

Although Bcl-w is a pro-survival Bcl-2 protein, the general location of the C-terminus is most similar to that seen for the pro-apoptotic protein Bax (Suzuki et al., 2000) (FIGS. 2B and 2C). While, the C-terminal residues in both proteins occupy the hydrophobic groove formed by residues from α2-α5 a detailed comparison reveals a number of differences. The C-terminal tail of Bax is shorter and forms a single α-helix, that lies in the centre of the hydrophobic groove (FIG. 2C). In contrast, the region beyond α8 in Bcl-wΔC10 is considerably longer and, unlike the continuous helix seen in Bax, only residues 157 to 173 have a helical conformation. Beyond α9 in Bcl-w is a region of irregular structure (residues 174-183) containing a number of hydrophobic residues (V173, L174, A177, V178, A179 and L180) (FIG. 2A). Contacts between these hydrophobic residues and those in α4 and the α4-α5 loop represent the main interactions that stabilise the C-terminus in Bcl-w. This differs from the situation in Bax (Suzuki et al., 2000), where the location and detailed interactions of their C-terminal residues with the BH1-3 groove differ. BH3 binding can readily displace the tail of Bcl-w to trigger tight membrane association and its inactivation (here and in Wilson-Annan et al., 2003.), but it is unclear if a similar mechanism operates to activate Bax (Cheng et al., 2001). Intriguingly, Bax translocation and consequent oligomerization, steps critical in its activation, appears to be linked to its C-terminal residues (Nechushtan et al., 1999; Suzuki et al., 2000). Thus understanding at atomic level how the C-terminal tails of pro-survival Bcl-w and pro-apoptotic Bax are regulated may be important for understanding their opposing biological activities.

Structural comparison of Bcl-wΔC10 and Bcl-x_{L}ΔC24 in complex with BH3 peptides from either Bak or Bad (rmsd of 1.58 Å over Cα, N, C' atoms of helices α1-α7 between Bcl-wΔC10 and Bcl-x_{L}ΔC24) (FIGS. 2B and 2C) suggests a mechanism for regulating the position of the C-terminus. The notable feature of this comparison is the similar locations of the BH3-domain peptide ligand and the C-terminus of Bcl-w, although they have opposite orientations with respect to the direction of the protein chain (FIG. 2C). The C-terminus of Bcl-x_{L}ΔC24 is truncated and displaced from the core of the protein. In contrast, the helices forming the hydrophobic groove (α2-α5) have very similar positions in Bcl-wΔC10 and the Bcl-x_{L}ΔC24 complex structures (FIG. 2C). In particular, when the structures are superimposed to give the overall best agreement α2, α4 and α5 overlay closely (rmsd 1.14 Å over Cα, N, C' atoms) and many of the corresponding side chains that contact the ligand in Bcl-x_{L}ΔC24 have similar rotamer conformations in either structure. Only in α3 are differences in the position of the helices and the associated side chains seen. Thus, binding of BH3-only ligands to Bcl-w probably requires displacement of the C-terminus from the groove but only small local movements of interacting residues. Once displaced, the hydrophobic C-terminal tail of Bcl-w would be free to bind membranes tightly.

### THE C-TERMINI OF BCL-W INFLUENCES BH3-DOMAIN BINDING

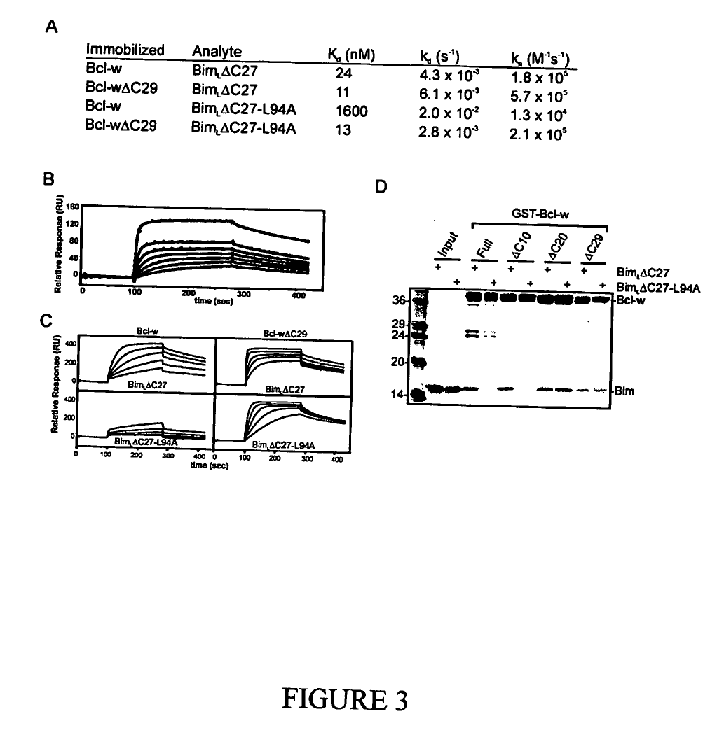

To test the idea that the C-terminus of Bcl-w might restrict access to the hydrophobic groove we examined the ability of Bim to interact with Bcl-w. The BH3-only protein Bim, is a potent initiator of apoptotic cell death and all isoforms, including Bim_{L} and Bim_{EL} used here, have identical BH3 domains (O'Connor et al., 1998). The BH3 domain, present in all the BH3-only proteins, is necessary for binding to and neutralising pro-survival Bcl-2 proteins, and in Bim mutation of a highly conserved residue in this domain (L94A) reduced binding to Bcl-w (or other pro-survival molecules) and killing activity (FIG. 3 and not shown). Purified, C-terminally truncated Bim_{L} (Bim_{L}ΔC27), and a mutant version (Bim_{L}ΔC27-L94A), were used to examine the binding properties of Bcl-w by surface plasmon resonance measurement on a BIAcore optical biosensor and by GST pull-down experiments.

As expected from other studies (O'Connor et al., 1998), biosensor and pull-down experiments demonstrated tight binding of wild-type Bim to Bcl-w (FIG. 3). Global analysis of the BIAcore binding data revealed unambiguously 1:1 Langmuir interactions between surface-immobilised Bim proteins and Bcl-w proteins in solution (FIG. 3B). This analysis also suggested a nanomolar affinity for the interaction between full length Bcl-w and Bim_{L}ΔC27 (K_{d} 24 nM). In contrast, Bim containing the L94 mutation bound with significantly reduced affinity (K_{d} 1600 nM) and the over 60-fold reduction was due to both, a 10-fold decrease in the association rate and a five-fold increase in the dissociation rates of the mutant protein (FIG. 3A). In agreement with the notion that the C-terminal region of Bcl-w restricts access of BH3 domains from interacting proteins, we observed a three-fold increase in the association rate between Bim_{L}ΔC27 and Bcl-w truncated by 29 residues at the C-terminus (Bcl-wΔC29), resulting in an improved affinity for this interaction (K_{d} 11 nM) (FIGS. 3B and 3C). In contrast to the results obtained with full-length protein, Bcl-wΔC29 also bound Bim_{L}ΔC27-L94A with a comparable affinity (K_{d} 13 nM). Similar results were obtained when a corresponding mutation (L138A) was introduced into another BH3-only protein, Bmf (not shown). The significantly increased affinity of the Bim and Bmf BH3 point mutants for the truncated Bcl-w proteins further supported a role for the C-terminus in regulating the binding of BH3-only proteins to Bcl-w.

To identify the C-terminal region that occluded the binding groove in Bcl-w, the ability of Bcl-w, or C-terminal truncations of it, to bind wild-type Bim_{L} or the L94A mutant was assessed by GST pull-down experiments (FIG. 3D). Bcl-wΔC10 behaved like full-length Bcl-w and only bound wt Bim_{L}ΔC27, whereas Bcl-wΔC20 and Bcl-wΔC29 bound equally well to Bim_{L}ΔC27 and Bim_{L}ΔC27-L94A (FIG. 3D). This suggests that residues 173-183, those that distinguish ΔC10 from ΔC20, have an essential role in preventing the interaction of Bim_{L}ΔC27-L94A with Bcl-w. This data is consistent with the NMR structure as these residues make a number of contacts with residues in the hydrophobic groove that are likely to stabilise the location of the C-terminus in Bcl-w (FIG. 2A). The comparable binding properties of full-length Bcl-w and Bcl-wΔC10 also suggest that these two proteins are likely to have similar structures and that the C-terminal residues restrict the interaction of some proteins with full-length Bcl-w.

### Access to the Surface Groove of Bcl-w and Bcl-x_{L} is Normally

### Restricted In Vivo

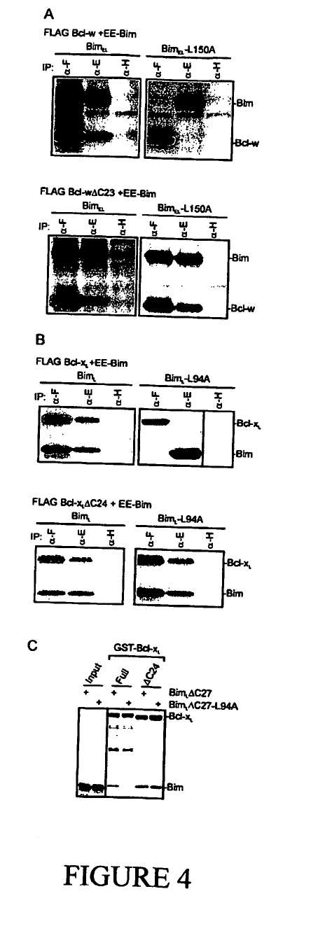

The structural and binding studies described herein suggest that access to the hydrophobic groove on Bcl-w may be normally restricted by its C-terminus. To determine if Bcl-w adopts a similar conformation in vivo we tested the ability of N-terminally FLAG-tagged full-length or C-terminally truncated Bcl-w, to bind to EE-tagged Bim when overexpressed in 293T cells. Depending on which could be more readily distinguished by its size, either Bim_{L} or Bim_{EL}, were used in these experiments. Interactions between wild-type or mutant Bim (L94A in Bim_{L} or L150A Bim_{EL}) and Bcl-w were measured by the ability of these proteins to be co-immunoprecipitated from 293T cell lysates (FIG. 4A). In agreement with the present findings with purified recombinant proteins (FIG. 3), Bcl-wΔC23 bound both wild-type and the L150A mutant Bim_{EL} equally, while full-length Bcl-w only bound wild-type Bim_{EL} (FIG. 4A).

Bcl-x_{L} and Bcl-2 also contain hydrophobic residues at their C-termini, similar to those found in Bcl-w (FIG. 1C), yet the low level of sequence identity following α8 and the absence of 3D-structural information, makes prediction of their conformation difficult. Since BCl-x_{L}, like Bcl-w, is only partially membrane-bound in healthy cells (Hsu et al., 1997), we compared the ability of FLAG-tagged full-length Bcl-x_{L} or a C-terminally truncated mutant (ΔC24) to bind to Bim_{L} or the □L94A B3 mutant. Like Bcl-w, full-length Bcl-x_{L} associates only with wild-type Bim_{L} in cell extracts (FIG. 4B) and more tightly with wild-type Bim_{L} in GST pull-down experiments (FIG. 4C). However, Bcl-x_{L}ΔC24 behaved like Bcl-wΔC29 since it bound wild-type Bim_{L} and the L94A mutant equally. Given that Bcl-x_{L} also becomes tightly associated with the membranes in response to apoptotic stimuli, presumably due to binding of BH3-only proteins as suggested for Bcl-w, the present results suggest similar roles for the C-terminal residues in both proteins.

### BH3-Binding is Insufficient for the Pro-Survival Activity of Bcl-w

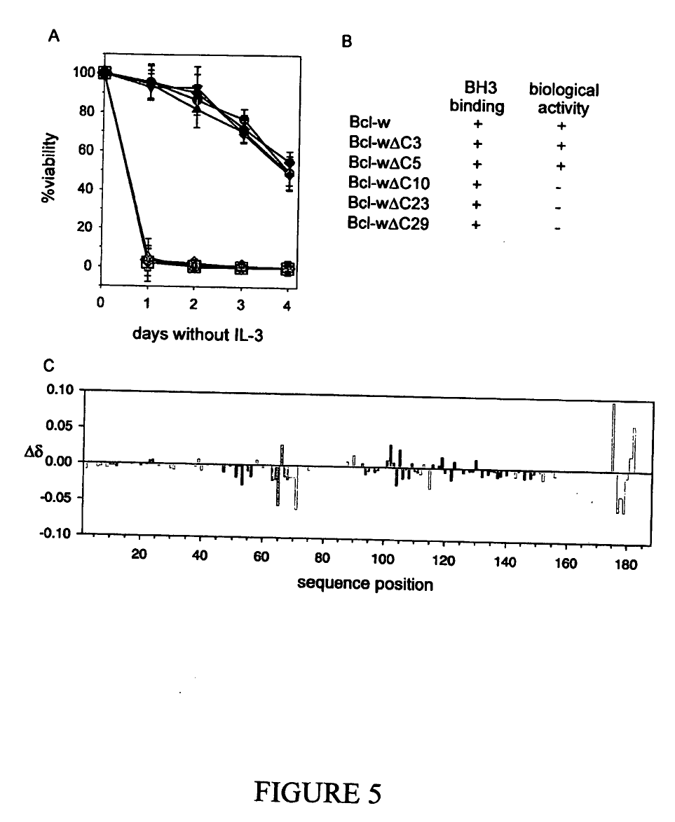

Like its cousins Bcl-2 and Bcl-x_{L}, Bcl-w overexpression protects cells from diverse death stimuli, including cytokine deprivation and γ-irradiation (Gibson et al., 1996). Since C-terminal truncation of Bcl-w did not affect binding to BH3-only proteins such as Bim, we explored whether BH3-binding alone is sufficient for the pro-survival activity of Bcl-w by comparing the functionality of full-length Bcl-w with C-terminal truncated variants (Bcl-w: -ΔC29; -ΔC23; -ΔC20; -ΔC5; and -ΔC3) when overexpressed in FDC-P1 myeloid cells. The survival of cells expressing comparable levels of FLAG-tagged proteins in response to IL-3 withdrawal, γ-irradiation, or cytotoxic drugs was monitored (FIGS. 5A and 5B). Surprisingly, only the smallest deletions (ΔC5 and ΔC3) were fully active. Expression of the other deletion mutants failed to afford the cells any protection, even though they were indistinguishable from full-length Bcl-w in their ability to bind to the BH3-only proteins Bim, Bad, Bik/Nbk or Bmf (FIGS. 3, 4 and not shown).

As Bcl-wΔC10 appeared biologically inert, while Bcl-wΔC5 behaved like the full-length protein, we next compared the spectra of these two molecules to determine if there was a structural basis for the marked functional difference. The ¹H-¹⁵N-HSQC spectra for Bcl-wΔC5 (A128E), the longest protein that we could purify in sufficient quantities for NMR analysis, was compared with that of Bcl-wΔC10 (FIG. 5C). Only small differences in the position of resonances were seen, consistent with addition of 5 residues to the C-terminus of Bcl-wΔC10. In addition, analysis of a ¹⁵N-edited NOESY spectrum obtained for Bcl-wΔC5 indicated that the additional 5 residues were disordered and all other residues that were ordered in Bcl-wΔC10 had a similar pattern of NOEs. Thus, despite its impaired biological activity, Bcl-wΔC10 is structurally equivalent to the functional Bcl-wΔC5 molecule (FIGS. 5A and 5C). Together the present findings demonstrate that the solution structure of Bcl-w reported here, which shows important differences from that of Bcl-2 and Bcl-x_{L} (Muchmore et al., 1996; Petros et al., 2001), is that of a biologically relevant molecule and represents the most complete model of a pro-survival Bcl-2 protein.

Since the structure and binding properties of functional Bcl-wΔC5 and non-functional Bcl-wΔC10 appear indistinguishable, we are currently investigating other possible differences between these proteins to explain their contrasting activities. One possible explanation is their localisation. Full-length Bcl-w is located exclusively on the outer mitochondrial membrane and as the C-terminal residues are important for localisation of other Bcl-2 proteins it is possible that in Bcl-w the most C-terminal residues have a critical role in mediating this association.

### Experimental Procedures

### Production of Bcl-w and Bim proteins

Human Bcl-w and mouse Bim_{L} were expressed as glutathione-S-transferase (GST) fusion proteins in E. coli BL21(DE3) and purified by affinity chromatography using Glutathione Sepharose (APB; Amersham Pharmacia Biotech). Following purification of the GST-fusion proteins Bcl-w and Bim were released from GST using PreScission protease (APB) and then further purified by size exclusion chromatography using a Superdex-75 column. All the purified proteins have 5 additional N-terminal residues (GPLGS) as a result of cloning. Isotopically labelled proteins were prepared as described previously (Day et al., 1999). Samples of Bcl-wΔC10 used for NMR contained ˜1.0 mM protein in 50 mM sodium phosphate (pH 6.7), 70 mM NaCl, 2 mM tris-(2-carboxyethyl) phosphine (TCEP) and 0.04% sodium azide in H₂O:²H₂O (95:5). The Bcl-wΔC5 sample contained 0.4 mM protein in the same buffer. Site specific mutants of Bim_{L} and Bcl-w were generated using a PCR based strategy as described previously (Day et al., 1999). The sequence of all clones was confirmed by sequencing.

### NMR Spectroscopy and Spectral Assignments

Spectra were recorded at 30° C. on a Bruker DRX-600 spectrometer equipped with triple resonance probes and pulsed field gradients. A series of heteronuclear 3D NMR experiments were recorded using either ¹⁵N or ¹³C, ¹⁵N double labelled protein (Sattler et al., 1999). Experiments recorded on ¹⁵N-labeled Bcl-wΔC10 included a ¹⁵N-edited NOESY-HSQC at mixing times of 50 and 150 ms, HSQC, HNHA, ¹⁵N-edited TOCSY-HSQC. Triple resonance experiments recorded on a ¹³C, ¹⁵N-labeled Bcl-wΔC10 sample included a HNCA, CBCA(CO)NH and ¹³C-edited NOESY-HSQC at mixing times of 50 and 150 ms, a 3D HCCH-TOCSY was also recorded on this sample. A 150 ms mixing time 2D NOESY was acquired on unlabeled Bcl-wΔC10. A ¹⁵N HSQC and 150 ms ¹⁵N edited NOESY-HSQC were recorded on ¹⁵N-Bcl-wΔC5. Spectra were referenced relative to DSS in the ¹H dimension and according to Wishart et al. (1995) in the ¹³C or ¹⁵N dimension, processed using XWINNMR (Bruker A G) and analyzed using XEASY (Bartels et al., 1995).

### Distance And Dihedral Angle Restraints

Distance restraints were measured from the 3D ¹⁵N-edited NOESY-HSQC, 3D ¹³C-edited NOESY as well as the 2D NOESY spectra. Peak integration was performed using XEASY and the calculated distances were calibrated using the CALIBA protocol in DYANA (Güntert et al., 1997). Hydrogen bond constraints were applied at a late stage of the structure calculation where there existed the characteristic low ³J_{HNHα} coupling constant and NOE patterns observed for α-helices. For each hydrogen bond constraint, upper limits of 2.3 and 3.3 Å were used for the distances from proton to acceptor and donor nitrogen atom to acceptor, respectively. No hydrogen bond constraints were employed outside α-helices.

Dihedral angle restraints for φ, ψ, χ1 and χ2 angles were used as summarized in Table 1. ³J_{HNHα} were derived from a 3D HNHA spectrum (Vuister and Bax, 1993) and were converted to φangle restraints as follows: ³J_{HNHα}<5 Hz, φ=−60±25°, ³J_{HNHα<}6 Hz, φ=−60±30°³J_{HNHα}≧8 Hz, φ=−120±30°. Additional φ and ψ backbone torsion angles plus uncertainties for these values were derived from ¹³Cαchemical shifts or to negative φ angles where the condition for a positive φangle was not met according to procedures we have reported previously (Day et al., 1999). Stereospecific assignments, χ1 and χ2 restraints were derived using HABAS and GLOMSA routines in DYANA (Güintert et al., 1997).

### Structure Calculation and Analysis

Initial structures were calculated using DYANA 1.5 (Güntert et al., 1997) using the experimental distance and dihedral angle restraints with the torsion angle dynamics simulated annealing protocol. Structures were optimized in DYANA to obtain low target functions and once a final set of experimental constraints had been established a new family of structures was determined and refined with CNS 1.1 (Brünger et al., 1998). The final step in the structure determination protocol involved minimisation in a box of water with the OPLSX force field (Linge and Nilges, 1999) in CNS. Structural statistics for the final set of 20 structures, chosen on the basis of their stereochemical energies, are presented in Table 2. PROCHECK₁₃ NMR (Laskowski et al., 1996) and MOLMOL (Koradi et al., 1996) were used for the analysis of structure quality. The final structures had no experimental distance violations greater than 0.25 Å or dihedral angle violations greater than 5°. Structural figures were generated in MOLMOL.

### Binding Measurements

Direct interactions between Bcl-w and Bim_{L} were monitored using GST pull-down experiments. These were performed in 100 μl of PBS (phosphate buffered saline pH 7.3) containing 2 mM DTT. Typically excess soluble proteins were added to equivalent amounts of resin bound proteins. After incubation at room temperature for 30 minutes the resin was pelleted and then washed twice with 200 μl of PBS containing 0.2% Tween 20. After removal of the second wash 10 μl of SDS-PAGE sample buffer was added to each sample and the samples were boiled then loaded onto 16% polyacrylamide gels that were electrophoresed and stained with Coomassie Blue R250. The staining intensity of the bound soluble protein indicated the strength of the interaction between the two proteins.

Analysis of protein interactions by surface plasmon resonance was carried out on a BIAcore 2000 biosensor (BIAcore). For the immobilisation to BIAcore CM 5 sensorchips (BIAcore), wild-type or mutant Bim proteins were buffer exchanged into 20 mM Na-acetate, pH 4.5. N-hydroxysuccinimide coupling and binding analysis was done as described previously (Lackmann et al., 1997). In order to minimise mass-transport mediated effects the kinetic experiments were routinely carried out at 20 μl/min. Bcl-w binding at concentrations between 2 and 0.03 μM in running buffer (10 mM HEPES, pH 7.4, 150 mM NaCl, 3.4 mM EDTA, 0.005% Tween 20) was performed on sensorchip surfaces derivatised on parallel channels with a non-relevant protein, Bim_{L}ΔC27 and Bim_{L}ΔC27-L94A. The binding kinetics were derived from the sensorgrams following subtraction of baseline responses (measured on the control channel) by ‘global analysis’ using the BIA Evaluation software (vers. 3.02, BIAcore). The surface of the chip was regenerated with 50 mM 1,2-diethylamine containing 0.1% Triton X100, followed by two washes with running buffer.

### Tissue Culture, Transfection and Immunoprecipitation

Cell culture, stable transfections into FDC-P1, transient transfections into 293T human embryonic kidney cells, metabolic labelling with ³⁵S-methionine/cysteine (NEN) and co-immunoprecipitation have been previously described (O'Connor et al., 1998, Moriishi et al., 1999; Huang et al., 1998; Wilson-Annan et al., 2003). Briefly, mammalian pEF-based expression vectors for Bcl-w, BCl-x_{L}, Bim, Bmf, Bad and Bik (O'Connor et al., 1998, Huang et al., 1998; Moriishi et al., 1999; Wilson-Annan et al., 2003) were transiently transfected using liposome mediated transfection (Lipofectamine™; Invitrogen). 48 hours after transfection, equivalent trichloroacetic acid (TCA; Sigma)-precipitable ³⁵S counts were immunoprecipitated using the anti-FLAG M2 (Sigma), anti-EE (Glu-Glu) (BabCo) or anti-HA.11 (BabCo) mouse monoclonal antibodies. The immunoprecipitates were resolved by SDS-PAGE (Novex), transferred onto nitrocellulose membranes (APB) and the proteins detected by fluorography (Amplify; APB).

### Survival Assays

Survival assays were performed as described previously (O'Connor et al., 1998; Huang et al., 1998; Wilson-Annan et al., 2003; and references therein). Briefly, cells (2−5×10⁴ per time point) were left untreated, deprived of their essential growth factor IL-3, exposed to 10Gy γ-irradiation (provided by a ⁶⁰Co source), 1-100 nM staurosprorine (Sigma). Cell viability was quantified by flow cytometric analysis of cells excluding 5 μg/mL PI (Sigma) using a FACScan (Becton Dickinson). Each time point was performed in triplicate on at least 3 independent clones of each genotype and the experiments repeated at least 3 times.

### Assaying the Action of Compounds Against Bcl-w, Bcl-2 and Bcl-x_{L}

### In Vitro Binding Assays

### NMR

NMR methods can be used to screen compounds by examination of perturbations in the resonance positions of the protein, relaxation properties or translational diffusion rates of the ligand (Stockman and Dalvit 2002). It may also be used to determine binding constants where they are low (Kd ˜>20 μM).

Specific binding of small compounds to Bcl-w will be monitored using ¹⁵N-labelled Bcl-w to look for changes in resonance position of residues in the BH3-peptide binding. Compounds will be titrated into solutions of ¹⁵N Bcl-w and the chemical shifts of the amide resonances monitored. Where the compound is in fast exchange (weak binding) Kd values can be extracted from the titration curve, while in the case of slow exchange (tight binding) a structure for the Bcl-w ligand complex can be determined. Such a method can be extended to look for chemical shift perturbations in Bcl-2 and Bcl-x_{L}.

Chemical shift monitoring can be used to screen compounds discovered in any computational (in silico) screen that have potential binding to Bcl-w (or suitably labelled Bcl-2 or Bcl-x_{L}), in this procedure mixtures of compounds are aided and then deconvoluted if a binding event, as measured by a chemical shift perturbation is observed in the protein resonances.

### Optical Biosensor

The BIAcore instrument can be used to measure the binding of small molecules directly or in a competition binding mode, where a much larger ligand (such as Bim) is displaced from Bcl-w (Malmqvist, 1999).

### In Vivo Assay

### Cell Based

Promising lead compounds will be subjected to a thorough analysis of their efficacy in killing a variety of cell lines and in mouse tumour models. Their activity on cell viability will be assessed on a panel of cultured tumorigenic and non-tumorigenic cell lines, as well as primary mouse or human cell populations, e.g. lymphocytes. Cell viability and total cell numbers will be monitored over 3-7 days of incubation with 1 nM-100 μM of the compounds to identify those that kill at IC50<10 μM.

Such compounds will be evaluated for the specificity of their targets and mode of action in vivo. For example, if a lead compound binds with high selectivity to Bcl-2, it should not kill cells lacking Bcl-2. Hence, the specificity of action can be confirmed by comparing the activity of the compound in wild-type cells with those lacking Bcl-2, derived from Bcl-2-deficient mice.

### Animal Models

To assess the anti-tumour efficacy of potential BH3 mimetics in vivo, the BH3 mimetics will either be given alone (intra-venously; iv or intra-peritoneally; ip) or in combination with sub-optimal doses of clinically relevant chemotherapy (e.g. 25-100 mg/kg cyclophosphamide intra-peritoneally). Mice injected intra-peritoneally with 10⁶ Bcl-2-overexpressing mouse lymphoma cells (Strasser 1996; Adams 1999) develop an aggressive immature lymphoma that is rapidly fatal within 4 weeks if untreated, but are partially responsive to cyclophosphamide. The lymphoma/leukaemia can readily be monitored by performing peripheral blood counts in the animals using a Coulter counter or by weighing the lymphoid organs (lymph nodes, spleen) when the animals are sacrificed. Another model is implantation of a cell line such as that derived from human follicular lymphoma (DoHH2) into immunocompromised SCID mice (Lapidot 1997). Because the BH3 mimetics might prove most efficacious in combination therapy, their in vivo activity will be evaluated alone or in combination with conventional chemotherapeutic agents (e.g. cyclophosphamide, doxorubucin, epipodophylotoxin (etoposide; VP-16)). Cohorts of 18-20 mice per treatment arm will be studied to enable a 25% difference in efficacy with a power of 0.8 at a significance level of 0.05 to be determined. These in vivo tests in mice will also generate preliminary pharmacokinetic, pharmacodynamic and toxicology data.

In parallel with these studies, extended analysis of selected compounds (e.g. those killing at <10 μM) will be undertaken through the gratis services of the National Cancer Institute (NCI) Developmental Therapeutics Program. It conducts tests on submitted compounds for chemotherapeutic activity against a panel of 60 human tumour cell lines (including leukemias). If useful potency is revealed, the inventors will undertake in vivo tests for both anti-tumour activity and toxicity on 12 human tumour lines growing in hollow fibers implanted into athymic mice.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

LENGTHY TABLE REFERENCED HERE
US20070054846A1-20070308-T00001
Please refer to the end of the specification for access instructions.

LENGTHY TABLE REFERENCED HERE
US20070054846A1-20070308-T00002
Please refer to the end of the specification for access instructions.

LENGTHY TABLE REFERENCED HERE
US20070054846A1-20070308-T00003
Please refer to the end of the specification for access instructions.

### BIBLIOGRAPHY

Adams, J. M., Cory, S. (1992). Oncogene cooperation in leukemogenesis. Cancer Surv. 15, 119-141.
Adams, J. M., Cory, S. (1998). The Bcl-2 protein family: arbiters of cell survival. Science 281, 1322-1326.
Adams, J. M., A. W. Harris, A. Strasser, S. Ogilvy, and S. Cory. (1999). Transgenic models of lymphoid neoplasia and development of a pan-hematopoietic vector. Oncogene 18, 5268.
Adams, J. M., Cory, S. (2001). Life-or-death decisions by the Bcl-2 protein family. Trends Biochem. Sci., 26: 61-66.
Andrews, M. J. I., Tabor, A. M. (1999). Forming stable helical peptides using natural and artificial amino acids. Tetrahedron 55, 11711-11743.
Ashkenazi, A, Dixit, V. M. (1998). Death receptors: signaling and modulation. Science 281, 1305-1308.
Bartels, C., Xia, T. H., Billeter, M., Güntert, P., and Wüthrich, K. (1995). The Program XEASY for Computer-Supported NMR Spectral-Analysis of Biological Macromolecules. J. Biomol. NMR 6, 1-10.
Borner, C., Martinou, I., Mattmann, C., Irmler, M., Schaerer, E., Martinou, J. C., and Tschopp, J. (1994). The protein Bcl-2 alpha does not require membrane attachment, but two conserved domains to suppress apoptosis. J. Cell Biol. 126, 1059-1068.
Bouillet, P., Metcalf, D., Huang, D. C. S., Tarlinton, D. M., Kay, T. W., Kontgen, F., Adams, J. M., and Strasser, A. (1999). Proapoptotic Bcl-2 relative Bim required for certain apoptotic responses, leukocyte homeostasis, and to preclude autoimmunity. Science 286, 1735-1738.
Brown J M, Wouters B G, (1999). Apoptosis, p53, and tumor cells sensitivity to anticancer agents. Cancer Res. 59, 1391-1399.
Brünger, A. T., Adams, P. D., Clore, G. M., DeLano, W. L., Gros, P., Grosse-Kunstleve, R. W., Jiang, J. S., Kuszewski, J., Nilges, M., Pannu, N. S., et al. (1998). Crystallography & NMR system: A new software suite for macromolecular structure determination. Acta Crystallogr. D. 54, 905-921.
Burgi R., Daura Z., Mark A., Bellanda M., Mamrnmi S, Peggion E., van Gunsteren W. (2001). Folding study of an Aib-rich peptide in DMSO by molecular dynamics simulations. J. Pept. Res. 57, 107-118.
Chen-Levy, Z., Nourse, J., and Cleary, M. L. (1989). The bcl-2 candidate proto-oncogene product is a 24-kilodalton integral-membrane protein highly expressed in lymphoid cell lines and lymphomas carrying the t(14;18) translocation. Mol. Cell. Biol. 9, 701-710.
Chen, F, Hersh, B. M., Conradt, B, Zhou, Z., Reimer, D., Gruenbaum, Y., Horvitz, H. R. (2000). Translocation of C. elegans CED4 to nuclear membranes during programmed cell death. Science 287, 1485-1489.
Chen, L., Wright L. R., Chen C H, Oliver S F, Wender P A, Mochly-Rosen D. (2001). Molecular transporters for peptides: delivery of a cardioprotective ePKC agonist peptide into cells and intact ischemic heart using a transport system. Chem. Biol. 8, 1123-1129.
Cheng, E. H., Wei, M. C., Weiler, S., Flavell, R. A., Mak, T. W., Lindsten, T., and Korsmeyer, S. J. (2001). Bcl-2, Bcl-x_{L} sequester BH3 domain-only molecules preventing BAX- and BAK-mediated mitochondrial apoptosis. Mol. Cell. 8, 705-711.
Chin J W, Schepartz A. (2001). Design and evolution of a miniature Bcl-2 binding protein. Angew. Chem. Int. Ed. 40, 3806-3809.
Chinnaiyan A M, O'Rourke K, Lane B R, Dixit V M. (1997). Interaction of CED-4 with CED-3 and CED-9: a molecular framework for cell death. Science 275, 1122-1126.
Chou, J. J., Li, H., Salvesen, G. S., Yuan, J., and Wagner, G. (1999). Solution structure of BID, an intracellular amplifier of apoptotic signaling. Cell 96, 615-624.
Christopher Phelan J, Skelton N J, Braisted A C, McDowell R S. (1997). A general method for constraining short peptides to an alpha-Helical conformation. J. Am. Chem. Soc. 119, 455-460.
Clearly, ML, Smith, S D, Sklar, J. (1986). Cloning and structural analysis of cDNAs for Bcl-2 and a hybrid Bcl-2/immunoglobulin transcript resulting from the t(14;18) translocation. Cell 47, 19-28.
Conus, S. Rossé, T. Borner C. (2000). Failure of Bcl-2 family members to interact with Apaf-1 in normal and apoptotic cells. Cell Death Differ. 7, 947-954.
Cory, S., and Adams, J. M. (2002). The Bcl-2 family: Regulators of the cellular life-or-death switch. Nature Reviews Cancer. 2, 647-677.
Day, C. L., Dupont, C., Lackmann, M., Vaux, D. L., and Hinds, M. G. (1999). Solution structure and mutagenesis of the caspase recruitment domain (CARD) from Apaf-1. Cell Death Differ. 6, 1125-1132.
del Mar Martinez-Senac, M., Corbalan-Garcia, S., and Gomez-Fernandez, J. C. (2000). Study of the secondary structure of the C-terminal domain of the antiapoptotic protein Bcl-2 and its interaction with model membranes. Biochemistry 39, 7744-7752.
Fairlie D P, West M L, Wong A k. (1998). Towards protein surface mimetics. Curr. Med. Chem. 5, 29-62.
Fisher, D. E. (1994). Apoptosis in cancer therapy: crossing the threshold. Cell 78, 539-542.
Gibson, L., Holmgreen, S. P., Huang, D. C. S., Bernard, O., Copeland, N. G., Jenkins, N. A., Sutherland, G. R., Baker, E., Adams, J. M., and Cory, S. (1996). Bcl-w, a novel member of the Bcl-2 family, promotes cell survival. Oncogene 13, 665-675.
Gonzalez-Garcia, M., Perez-Ballestero, R., Ding, L., Duan, L., Boise, L. H., Thompson, C. B., and Núñez, G. (1994). bcl-x_{L} is the major bcl-x mRNA form expressed during murine development and its product localizes to mitochondria. Development 120, 3033-3042.
Gras-Masse H. (2001). Single-chain lipopeptide vaccines for the induction of virus-specific cytotoxic T cell responses in randomly selected populations. Mol. Immunol. 38, 423-431.
Güntert, P., Mumenthaler, C., and Wüthrich, K. (1997). Torsion angle dynamics for NMR structure calculation with the new program DYANA. J. Mol. Biol. 273, 283-298.
Han J, Flemington C. Houghton A B, Gu Z, Zambetti G P, Lutz R J, Zhu L, Chittenden T. (2001). Expression of bbc3, a pro-apoptotic BH3-only gene, is regulated by diverse cell death and survival signals. Proc. Natl. Acad. Sci. USA 98, 11318-11323.
Hausmann, G., O'Reilly, L. A., van Driel, R., Beaumont, J. G., Strasser, A., Adams, J. M., and Huang, D. C. S. (2000). Pro-apoptotic apoptosis protease-activating Factor 1 (Apaf-1) has a cytoplasmic localization distinct from Bcl-2 or Bcl-x_{L}. J. Cell Biol. 149, 623-634.
Haraguchi M, Torii S, Matsuzawa S, Xie Z, Kitada S, Krajewski S, Yoshida H, Mak T W, Reed J C. (2000). Apoptotic protease activating factor 1 (Apaf-1)-independent cell death suppression by Bcl-2. J. Exp. Med. 191, 1709-1720.
Holinger E P, Chittenden T, Lutz R J. (1999). Bak BH3 peptides antagonize Bcl-x_{L} funciton and induce apoptosis through cytochrome c-independent activation of caspases. J. Biol. Chem. 274, 13298-13304.
Hsu, Y. T., Wolter, K. G., and Youle, R. J. (1997). Cytosol-to-membrane redistribution of Bax and Bcl-X(L) during apoptosis. Proc. Natl. Acad. Sci. USA 94, 3668-3672.
Huang, D. C. S., Adams, J. M., and Cory, S. (1998). The conserved N-terminal BH4 domain of Bcl-2 homologues is essential for inhibition of apoptosis and interaction with CED4. EMBO J. 17, 1029-1039.
Huang, D. C. S., and Strasser, A. (2000). BH3-Only proteins-essential initiators of apoptotic cell death. Cell 103, 839-842.
Huang, Q., Petros, A. M., Virgin, H. W., Fesik, S. W., and Olejniczak, E. T. (2002). Solution structure of a Bcl-2 homolog from Kaposi sarcoma virus. Proc. Natl. Acad. Sci. USA 99, 3428-3433.
Judice J K, Tom J Y, Huang W, Wrin T, Vennari J, Petropoulos C J, McDowell RS. (1997). Inhibition of HIV type I infectivity by constrained alpha-helical peptides: implications for the viral fusion mechanism. Proc. Natl. Acad. Sci. USA 94, 13426-13430.
Kasher R, Oren D A, Barda Y, Gilon C. (1999). Miniaturized proteins: the backbone cyclic proteinomimetic approach. J. Mol. Biol. 292, 421-429.
Kleiger, G., Grothe, R., Mallick, P., and Eisenberg, D. (2002). GXXXG and AXXXA: common alpha-helical interaction motifs in proteins, particularly in extremophiles. Biochemistry 41, 5990-5997.
Koradi, R., Billeter, M., and Wüthrich, K. (1996). MOLMOL: A program for display and analysis of macromolecular structures. J. Mol. Graph. 14, 51-55.
Krajewski, S., Tanaka, S., Takayama, S., Schibler, M. J., Fenton, W., and Reed, J. C. (1993). Investigation of the subcellular distribution of the Bcl-2 oncoprotein: residence in the nuclear envelope, endoplasmic reticulum, and outer mitochondrial membranes. Cancer Res. 53, 4701-4714.
Lackmann, M., Mann, R. J., Kravets, L., Smith, F. M., Bucci, T. A., Maxwell, K. F., Howlett, G. J., Olsson, J. E., Vanden Bos, T., Cerretti, D. P., and Boyd, A. W. (1997). Ligand for EPH-related kinase (LERK) 7 is the preferred high affinity ligand for the HEK receptor. J. Biol. Chem. 272, 16521-16530.
Lane D P. (1992). p53, guardian of the genome. Nature 358, 15-16
Lapidot, T., Y. Fajerman, and O. Kollet. (1997). Immune-deficient SCID and NOD/SCID mice models as functional assays for studying normal and malignant human hematopoiesis. Journal of Molecular Medicine 75, 664.
Laskowski, R. A., Rullmann, J. A. C., MacArthur, M. W., Kaptein, R., and Thornton, J. M. (1996). AQUA and PROCHECK-NMR: Programs for checking the quality of protein structures solved by NMR. J. Biomol. NMR 8, 477-486.
Linge, J. P., and Nilges, M. (1999). Influence of non-bonded parameters on the quality of NMR structures: A new force field for NMR structure calculation. J. Biomol. NMR 13, 51-59.
Lithgow, T., van Driel, R., Bertram, J. F., and Strasser, A. (1994). The protein product of the oncogene Bcl-2 is a component of the nuclear envelope, the endoplasmic reticulum, and the outer mitochondrial membrane. Cell Growth Differ. 5, 411-417.
Lowe S W, Schmitt E M, Smith S W, Osborne B A, (1993). Lacks T. p53 is required for radiation-induced apoptosis in mouse thymocytes. Nature 362, 847-849
Lu, G. G. (2000). TOP: a new method for protein structure comparisons and similarity searches. J. Appl. Cryst. 33, 176-183.
Malmqvist, M. (1999) BIACORE: an affinity biosensor system for characterization of biomolecular interactions. Biochem Soc Trans, 27, 335-340.
Marsden, V. S., O'Connor, L., O'Reilly, L. A., Silke, J., Metcalf, D., Ekert, P. G., Huang, D. C. S., Cecconi, F., Kuida, K., Tomaselli, K. J., Roy, S., Nicholson, D. W., Vaux, D. L., Bouillet, P., Adams, J. M. and Strasser, A. (2002) Apoptosis initiated by Bcl-2 regulated caspase activation independently of the cytochrome c/Apaf-1/caspase-9 apoptosome. Nature, 419, 634-637.
McDonnell, J. M., Fushman, D., Milliman, C. L., Korsmeyer, S. J., and Cowburn, D. (1999). Solution structure of the proapoptotic molecule BID: a structural basis for apoptotic agonists and antagonists. Cell 96, 625-634.
Moriishi, K., Huang, D. C. S., Cory, S., and Adams, J. M. (1999). Bcl-2 family members do not inhibit apoptosis by binding the caspase activator Apaf-1. Proc. Natl. Acad. Sci. USA 96, 9683-9688.
Muchmore, S. W., Sattler, M., Liang, H., Meadows, R. P., Harlan, J. E., Yoon, H. S., Nettesheim, D., Chang, B. S., Thompson, C. B., Wong, S. L., Ng, S. C., and Fesik, S. W. (1996). X-ray and NMR structure of human Bcl-x_{L}, an inhibitor of programmed cell death. Nature 381, 335-341.
Nakano K, Wousden K H. (2001). PUMA, a novel proapoptotic gene, is induced by p53. Mol. Cell. 7, 683-694.
Nechushtan, A., Smith, C. L., Hsu, Y. T., and Youle, R. J. (1999). Conformation of the Bax C-terminus regulates subcellular location and cell death. EMBO J. 18, 2330-2341.
Nechushtan, A., Smith, C. L., Lamensdorf, I., Yoon, S. H., and Youle, R. J. (2001). Bax and Bak coalesce into novel mitochondria-associated clusters during apoptosis. J. Cell Biol. 153, 1265-1276.
O'Connor, L., Strasser, A., O'Reilly, L. A., Hausmann, G., Adams, J. M., Cory, S., and Huang, D. C. S. (1998). Bim: a novel member of the Bcl-2 family that promotes apoptosis. EMBO J. 17, 384-395.
Oda E, Ohki R, Murasawa H, Nemoto J, Shibue T, Yamashita T, Tokino T, Taniguchi T, Tanaka N. (2000). Noxa, a BH3-only member of the Bcl-2 family and candidate mediator of p53-induced apoptosis. Science 288, 1053-1058.
O'Reilly, L. A., Print, C., Hausmann, G., Moriishi, K., Cory, S., Huang, D. C. S., and Strasser, A. (2001). Tissue expression and subcellular localization of the pro-survival molecule Bcl-w. Cell Death Differ. 8, 486-494.
Petros, A. M., Medek, A., Nettesheim, D. G., Kim, D. H., Yoon, H. S., Swift, K., Matayoshi, E. D., Oltersdorf, T., and Fesik, S. W. (2001). Solution structure of the antiapoptotic protein Bcl-2. Proc. Natl. Acad. Sci. USA 98, 3012-3017.
Petros, A. M., Nettesheim, D. G., Wang, Y., Olejniczak, E. T., Meadows, R. P., Mack, J., Swift, K., Matayoshi, E. D., Zhang, H., Thompson, C. B., and Fesik, S. W. (2000). Rationale for Bcl-x_{L}/Bad peptide complex formation from structure, mutagenesis, and biophysical studies. Protein Sci. 9, 2528-2534.
Print, C. G., Loveland, K. L., Gibson, L., Meehan, T., Stylianou, A., Wreford, N., de Kretser, D., Metcalf, D., Kontgen, F., Adams, J. M., and Cory, S. (1998). Apoptosis regulator Bcl-w is essential for spermatogenesis but appears otherwise redundant. Proc. Natl. Acad. Sci. USA 95, 12424-12431.
Puthalakath H, Huang D C S, O'Reilly L A, King S M, Strasser A. (1999). The pro-apoptotic activity of the Bc102 family member Bim is regulated by interaction with the dynein motor complex. Mol. Cell. 3, 287-296.
Puthalakath H, Villunger A, O'Reilly L A, Beaumont J G, Coultas L, Cheney R E, Huang D C S, Strasser A. (2001). Bmf: a pro-apoptotic BH3-only protein regulated by interaction with the myosin V actin motor complex, activated by anoikis. Science 293, 1829-1832.
Sattler, M., Liang, H., Nettesheim, D., Meadows, R. P., Harlan, J. E., Eberstadt, M., Yoon, H. S., Shuker, S. B., Chang, B. S., Minn, A. J., Thompson, C. B., and Fesik, S. W. (1997). Structure of Bcl-x_{L}-Bak peptide complex: recognition between regulators of apoptosis. Science 275, 983-986.
Sattler, M., Schleucher, J., and Griesinger, C. (1999). Heteronuclear multidimensional NMR experiments for the structure determination of proteins in solution employing pulsed field gradients. Prog. Nucl. Magn. Reson. Spectrosc. 34, 93-158.
Schafineister C E, Po J, Verdine G L. (2000). An All-hydrocarbon cross-linking system for enhancing the helicity and metabolic stability of peptides. J. Am. Chem. Soc. 122, 5891-5892.
Schimmer A D, Hedley D W, Chow S, Pham N A, Chakrabartty A, Bouchard D, Mak T W, Trus M R, Minden M D. (2001). The BH3 domain of BAD fused to the Antennapedia peptide induces apoptosis via its alpha helical structure and independent of Bcl-2. Cell Death Differ 8, 725-733.
Scholtz J M, Baldwin R L. (1992). The mechanism of α-helix formation by peptides. Annu. Rev. Biophys. Biomol. Struct. 21, 95-118.
Spector M S, Desnoyers S, Hoeppner D J, Hengartner M O. (1997). Interaction between the C. elegans cell-death regulators CED-9 and CED4. Nature 385, 653-665.
Stockman, B. J. and Dalvit, C. (2002) NMR Screening in drug discovery and drug design. Prog. NMR Spectrosc. 41, 187-231.
Senes, A., Gerstein, M., and Engelman, D. M. (2000). Statistical analysis of amino acid patterns in transmembrane helices: the GxxxG motif occurs frequently and in association with beta-branched residues at neighboring positions. J. Mol. Biol. 296, 921-936.
Strasser A, Harris A W, Bath M L, Cory S. (1990). Novel primitive lymphoid tumours induced in transgenic mice by cooperation between myc and Bcl-2. Nature 348, 331-333.
Strasser, A., A. G. Elefanty, A. W. Harris, and S. Cory. (1996). Progenitor tumours from Em-bcl-2-myc transgenic mice have lymphomyeloid differentiation potential and reveal developmental differences in cell survival. EMBO Journal 15, 3823.
Strasser A, Harris A W, Jacks T, Cor S. (1994) DNA damage can induce apoptosis in proliferating lymphoid cells via p53-independent mechanisms inhibitable by Bcl-2. Cell 79, 329-339.
Strasser A, O'Connor L, Dixit V M. (2000). Apoptosis signaling. Annu. Rev. Biochem 69, 217-245.
Suzuki, M., Youle, R. J., and Tjandra, N. (2000). Structure of Bax: coregulation of dimer formation and intracellular localization. Cell 103, 645-654.
Thompson C. B. (1995) Apoptosis in the pathogenesis and treatment of disease. Science 267, 1456-1462.
Thornberry N A, Lazebnik Y. (1998). Caspases: enemies within. Science 281, 1312-1316.
Tsujimoto Y, Cossman J, Jaffe E, Croce C M. (1985). Involvement of the Bcl-2 gene in human follicular lymphoma. Science 228, 1440-1443.
Vaux D L, Cory S, Adams J M. (1988). Bcl-2 gene promotes haemopoietic cell survival and cooperates with c-myc to immortalize pre-B cells. Nature 335, 440-442.
Vaux, D. L., and Korsmeyer, S. J. (1999). Cell death in development. Cell 96, 245-254.
Vousden K H. (2000). p53: death star. Cell 103, 691-694.
Vuister, G. W., and Bax, A. (1993). Quantitative J Correlation: A New Approach For Measuring Homonuclear Three-Bond J(H^{N}H^{α}) Coupling Constants in ¹⁵N-Enriched Proteins. J. Am. Chem. Soc. 115, 7772-7777.
Wang J L, Zhang Z J, Choksi S, Shan S, Lu Z, Croce C M, Alnemri E S, Komgold R, Huang Z. (2000). Cell permeable Bcl-2 binding peptides: a chemical approach to apoptosis induction in tumor cells. Cancer Res. 60, 1498-1502.
Wilson-Annan, J., O'Reilly, L. A., Crawford, S. A., Hausmann, G., Beaumont J. G., Pamma, L. P., Chen, L., Lackmann, M., Lithgow, T., Hinds, M. G., Day, C. L., Adams, J. M., Huang, D. C. (2003). Proapoptotic BH3-only proteins trigger membrane integration of prosurvival Bcl-w and neutralize its activity. J. Cell Biol. 162:877-887.
Wishart, D. S., Bigam, C. G., Yao, J., Abildgaard, F., Dyson, H. J., Oldfield, E., Markley, J. L., and Sykes, B. D. (1995). ¹H, ¹³C and ¹⁵N Chemical Shift Referencing in Biomolecular NMR. J. Biomol. NMR 6, 135-140.
Wu D Y, Wallen H D, Nuñez G. (1997). Interaction and regulation of subcellular localization of CED4 by CED-9. Science 275, 1126-1129.
Yang X, Chang H Y, Baltimore D. (1998). Essential role of CED4 oligomerization in CED-3 activation and apoptosis. Science 281, 1355-1357.
Yu J, Zhang L, Hwang P M, Kinzler K W, Vogelstein B. (2001). PUMA induces the rapid apoptosis of colorectal cancer cells. Mol. Cell. 7, 673-682.

Zong, W. X., Lindsten, T., Ross, A. J., MacGregor, G. R., and Thompson, C. B. (2001). BH3-only proteins that bind pro-survival Bcl-2 family members fail to induce apoptosis in the absence of Bax and Bak. Genes Dev. 15, 1481-1486.

LENGTHY TABLE
The patent application contains a lengthy table section. A copy of the table is available in electronic form from the USPTO web site (http://seqdata.uspto.gov/?pageRequest=docDetail&DocID=US20070054846A1). An electronic copy of the table will also be available from the USPTO upon request and payment of the fee set forth in 37 CFR 1.19(b)(3).

1. A solution comprising a molecule or molecular complex that comprises a Bcl-w active site defined by the structure coordinates of at least three Bcl-w amino acid residues selected from the group consisting of: Arg59, Asp63, Leu64, Gln67, Phe79, Val 82, Val 102 and Leu106 as set forth in TABLE 1, or a variant of the molecule or molecular complex, wherein the variant comprises an active site that has a root mean square deviation from the Ca atoms of the amino acid residues defining the Bcl-w active site of not more than 1.1 A.

2. A solution according to claim 1, wherein the active site is further defined by the structure coordinates of at least three Bcl-w amino acid residues selected from the group consisting of: Glu52, Arg56, Arg58, Glu85, Arg95 and Lys 113 as set forth in TABLE 1.

3. A solution according to claim 1, wherein the active site is defined by the structure coordinates of at least three Bcl-w amino acid residues selected from the group consisting of: Gln44, Ala45, Ala48, Ala49, Gly50, Glu52, Phe53, Arg56, Phe57, Arg58, Arg59, Asp63, Leu64, Ala66, Gln67, His69, Val70, Arg78, Phe79, Gln81, Val82, Ser83, Glu85, Leu86, Phe87, Gln88, Gly89, Gly90, Pro91, Asn92, Trp93, Gly94, Arg95, Val97, Phe99, Phe102, Leu106, Phe147, Thr148, Ala149, Leu150, Tyr151 and Gly152, as set forth in TABLE 1.

4. A solution according to claim 1, wherein the active site is defined by the structure coordinates of at least three Bcl-w amino acid residues selected from the group consisting of: Gln44, Ala45, Met46, Arg47, Ala48, Ala49, Gly50, Asp51, Glu52, Phe53, Glu54, Thr55, Arg56, Phe57, Arg58, Arg59, Thr60, Ser62, Asp63, Leu64, Ala65, Ala66, Gln67, Leu68, His69, Val70, Thr71, Ala75, Gln76, Gln77, Arg78, Phe79, Thr80, Gln81, Val82, Ser83, Asp84, Glu85, Leu86, Phe87, Gln88, Gly89, Gly90, Pro91, Asn92, Trp93, Gly94, Arg95, Leu96, Val97, Ala98, Phe99, Phe102, Gly103, Leu106, Trp137, Ser141, Glu146, Phe147, Thr148, Ala149, Leu150, Tyr151 and Gly152, as set forth in TABLE 1.

5. A solution according to claim 1, wherein the molecule or molecular complex further comprises the C-terminal region of Bcl-w.

6. A solution according to claim 5, wherein the molecule or molecular complex comprises the C-terminal helix (α9, residues 157-173) and extended region (residues 174-183) of Bcl-w.

7. A solution according to claim 1, wherein the molecule or molecular complex comprises a polypeptide that is distinguished from Bcl- w by the deletion of at least one amino acid residue at the C-terminus of Bcl-w.

8. A solution according to claim 7, wherein the polypeptide is further distinguished from Bcl-w by the substitution of at least one hydrophobic amino acid residue with a charged amino acid residue.

9. A solution according to claim 8, wherein the hydrophobic amino acid residue is Ala 128 and the charged amino acid residue is glutamate or modified form thereof.

10. A solution according to claim 7, wherein the polypeptide is a Bcl-w derivative that lacks the last 10 amino acid residues of Bcl-w and that has Ala128 substituted with a glutamate residue or modified form thereof.

11. A solution according to claim 7, wherein the polypeptide comprises the sequence set forth in SEQ ID NO:2.

12. A polypeptide that is distinguished from Bcl-w by the deletion of at least one amino acid residue from the C-terminus of Bcl-w.

13. A polypeptide according to claim 12, which is further distinguished from Bcl-w by the substitution of at least one hydrophobic amino acid residue with a charged amino acid residue.

14. A polypeptide according to claim 13, wherein the hydrophobic amino acid residue is Ala 128 and the charged amino acid residue is glutamate or modified form thereof.

15. A polypeptide according to claim 12, which is a Bcl-w derivative that lacks the last 10 amino acid residues of Bcl-w and that has Ala128 substituted with a glutamate residue or modified form thereof.

16. A polypeptide according to claim 12, which consists essentially of the sequence set forth in SEQ ID NO:2.

17. A polynucleotide comprising a sequence that encodes a polypeptide that is distinguished from Bcl-w by the deletion of at least one amino acid residue from the C-terminus of Bcl-w.

18. A polynucleotide according to claim 17, wherein the polypeptide is distinguished from Bcl-w by the substitution of at least one hydrophobic amino acid residue with a charged amino acid residue.

19. A vector comprising the polynucleotide of claim 17.

20. A host cell comprising the polynucleotide of claim 17.

21. A host cell comprising a vector comprising the polynucleotide of claim 17.

22. A data store comprising data representing the structure coordinates of Bcl-w amino acid residues and which are capable of being used by a computer system to generate a three-dimensional representation of a molecule or molecular complex comprising a Bcl-w active site defined by the structure coordinates of at least three Bcl-w amino acid residues selected from the group consisting of: Arg59, Asp63, Leu64, Gln67, Phe79, Val 82, Val 102 and Leu106 as set forth in TABLE 1, or a variant of the molecule or molecular complex, wherein the variant comprises an active site that has a root mean square deviation from the Ca atoms of the amino acid residues defining the Bcl-w active site of not more than 1.1 A.

23. A data store according to claim 22, wherein the active site is further defined by the structure coordinates of at least three Bcl-w amino acid residues selected from the group consisting of: Glu52, Arg56, Arg58, Glu85, Arg95 and Lysl 13 as set forth in TABLE 1.

24. A data store according to claim 22, wherein the active site is defined by the structure coordinates of at least three Bcl-w amino acid residues selected from the group consisting of: Gln44, Ala45, Ala48, Ala49, Gly50, Glu52, Phe53, Arg56, Phe57, Arg58, Arg59, Asp63, Leu64, Ala66, Gln67, His69, Val70, Arg78, Phe79, Gln81, Val82, Ser83, Glu85, Leu86, Phe87, Gln88, Gly89, Gly90, Pro91, Asn92, Trp93, Gly94, Arg95, Val97, Phe99, Phe102, Leu106, Phe147, Thr148, Ala149, Leu150, Tyr151 and Gly152, as set forth in TABLE 1.

25. A data store according to claim 22, wherein the active site is defined by the structure coordinates of at least three Bcl-w amino acid residues selected from the group consisting of: Gln44, Ala45, Met46, Arg47, Ala48, Ala49, Gly50, Asp51, Glu52, Phe53, Glu54, Thr55, Arg56, Phe57, Arg58, Arg59, Thr60, Ser62, Asp63, Leu64, Ala65, Ala66, Gln67, Leu68, His69, Val70, Thr71, Ala75, Gln76, Gln77, Arg78, Phe79, Thr80, Gln81, Val82, Ser83, Asp84, Glu85, Leu86, Phe87, Gln88, Gly89, Gly90, Pro91, Asn92, Trp93, Gly94, Arg95, Leu96, Val97, Ala98, Phe99, Phe102, Gly103, Leu106, Trp137, Ser141, Glu146, Phe147, Thr148, Ala149, Leu150, Tyr151 and Gly152, as set forth in TABLE 1.

26. A computer system having data representing structural coordinates of Bcl-w amino acid residues, the computer system being adapted to generate, on the basis of the data, a three-dimensional representation of a molecule or molecular complex comprising a Bcl-w active site that is defined by the structure coordinates of at least three Bcl-w amino acid residues selected from the group consisting of: Glu52, Arg56, Arg58, Glu85, Arg95 and Lys 113, as set forth in TABLE 1, or a variant of the molecule or molecular complex, wherein the variant comprises an active site that has a root mean square deviation from the Ca atoms of the amino acid residues defining the Bcl-w active site of not more than 1.1 A.

27. A computer system according to claim 26, wherein the active site is further defined by the structure coordinates of at least three Bcl-w amino acid residues selected from the group consisting of: Glu52, Arg56, Arg58, Glu85, Arg95 and Lys 113 as set forth in TABLE 1.

28. A computer system according to claim 26, wherein the active site is defined by the structure coordinates of at least three Bcl-w amino acid residues selected from the group consisting of: Gln44, Ala45, Ala48, Ala49, Gly50, Glu52, Phe53, Arg56, Phe57, Arg58, Arg59, Asp63, Leu64, Ala66, Gln67, His69, Val70, Arg78, Phe79, Gln81, Val82, Ser83, Glu85, Leu86, Phe87, Gln88, Gly89, Gly90, Pro91, Asn92, Trp93, Gly94, Arg95, Val97, Phe99, Phe102, Leu106, Phe147, Thr148, Ala149, Leu150, Tyr151 and Gly152, as set forth in TABLE 1.

29. A computer system according to claim 26, wherein the active site is defined by the structure coordinates of at least three Bcl-w amino acid residues selected from the group consisting of: Gln44, Ala45, Met46, Arg47, Ala48, Ala49, Gly50, Asp51, Glu52, Phe53, Glu54, Thr55, Arg56, Phe57, Arg58, Arg59, Thr60, Ser62, Asp63, Leu64, Ala65, Ala66, Gln67, Leu68, His69, Val70, Thr71, Ala75, Gln76, Gln77, Arg78, Phe79, Thr80, Gln81, Val82, Ser83, Asp84, Glu85, Leu86, Phe87, Gln88, Gly89, Gly90, Pro91, Asn92, Trp93, Gly94, Arg95, Leu96, Val97, Ala98, Phe99, Phe102, Gly103, Leu106, Trp137, Ser141, Glu146, Phe147, Thr148, Ala149, Leu150, Tyr 151 and Gly152, as set forth in TABLE 1.

30. A computer system for producing a three-dimensional representation of a molecule or molecular complex, the computer system comprising: (a) a data store including data representing the structure coordinates of Bcl-w amino acid residues defining a Bcl-w active site that is defined by the structure coordinates of at least three Bcl-w amino acid residues selected from the group consisting of: Glu52, Arg56, Arg58, Glu85, Arg95 and Lys 113, as set forth in TABLE 1, or structural coordinates having a root mean square deviation from the Ca atoms of those residues of not more than 1.1 A; (b) a processing means for processing the data to generate a three-dimensional representation of a molecule or molecular complex comprising the Bcl-w active site or similarly shaped homologous active site for display; and (c) a display means for displaying the three-dimensional representation.

31. A computer system according to claim 31, wherein the active site is further defined by the structure coordinates of at least three Bcl-w amino acid residues selected from Glu52, Arg56, Arg58, Glu85, Arg95 and Lysl 13 as set forth in TABLE 1.

32. A computer system according to claim 31, wherein the active site is defined by the structure coordinates of at least three Bcl-w amino acid residues selected from the group consisting of: Gln44, Ala45, Ala48, Ala49, Gly50, Glu52, Phe53, Arg56, Phe57, Arg58, Arg59, Asp63, Leu64, Ala66, Gln67, His69, Val70, Arg78, Phe79, Gln181, Val82, Ser83, Glu85, Leu86, Phe87, Gln88, Gly89, Gly90, Pro91, Asn92, Trp93, Gly94, Arg95, Val97, Phe99, Phe102, Leu106, Phe147, Thr148, Ala149, Leu150, Tyr151 and Gly152, as set forth in TABLE 1.

33. A computer system according to claim 31, wherein the active site is defined by the structure coordinates of at least three Bcl-w amino acid residues selected from the group consisting of: Gln44, Ala45, Met46, Arg47, Ala48, Ala49, Gly50, Asp51, Glu52, Phe53, Glu54, Thr55, Arg56, Phe57, Arg58, Arg59, Thr60, Ser62, Asp63, Leu64, Ala65, Ala66, Gln67, Leu68, His69, Val70, Thr71, Ala75, Gln76, Gln77, Arg78, Phe79, Thr80, Gln81, Val82, Ser83, Asp84, Glu85, Leu86, Phe87, Gln88, Gly89, Gly90, Pro91, Asn92, Trp93, Gly94, Arg95, Leu96, Val97, Ala98, Phe99, Phe102, Gly103, Leu106, Trp137, Ser141, Glu146, Phe147, Thr148, Ala149, Leu150, Tyr 151 and Gly152, as set forth in TABLE 1.

34. An analysis method, executed by a computer system, for evaluating the ability of a chemical entity to associate with a molecule or molecular complex comprising an active site, the method comprising the steps of: (a) generating a model of the active site using structure coordinates wherein the root mean square deviation between the structure coordinates and the structure coordinates of the Bcl-w amino acid residues defining a Bcl-w active site is not more than about 1.1 A, wherein the Bcl-w active site is defined by the structure coordinates of at least three Bcl-w amino acid residues selected from the group consisting of: Glu52, Arg56, Arg58, Glu85, Arg95 and Lys113, as set forth in TABLE 1; (b) performing a fitting operation between the chemical entity and the model of the active site; and (c) quantifying the association between the chemical entity and the active site model, based on the output of the fitting operation.

35. An analysis method according to claim 34, wherein the active site is further defined by the structure coordinates of at least three Bcl-w amino acid residues selected from the group consisting of: Glu52, Arg56, Arg58, Glu85, Arg95 and Lysl 13 as set forth in TABLE 1.

36. An analysis method according to claim 34, wherein the active site is defined by the structure coordinates of at least three Bcl-w amino acid residues selected from the group consisting of: Gln44, Ala45, Ala48, Ala49, Gly50, Glu52, Phe53, Arg56, Phe57, Arg58, Arg59, Asp63, Leu64, Ala66, Gln67, His69, Val70, Arg78, Phe79, Gln81, Val82, Ser83, Glu85, Leu86, Phe87, Gln88, Gly89, Gly90, Pro91, Asn92, Trp93, Gly94, Arg95, Val97, Phe99, Phe102, Leu106, Phe147, Thr148, Ala 149, Leu150, Tyr 151 and Gly152, as set forth in TABLE 1.

37. An analysis method according to claim 34, wherein the active site is defined by the structure coordinates of at least three Bcl-w amino acid residues selected from the group consisting of: Gln44, Ala45, Met46, Arg47, Ala48, Ala49, Gly50, Asp51, Glu52, Phe53, Glu54, Thr55, Arg56, Phe57, Arg58, Arg59, Thr60, Ser62, Asp63, Leu64, Ala65, Ala66, Gln67, Leu68, His69, Val70, Thr71, Ala75, Gln76, Gln77, Arg78, Phe79, Thr80, Gln81, Val82, Ser83, Asp84, Glu85, Leu86, Phe87, Gln88, Gly89, Gly90, Pro91, Asn92, Trp93, Gly94, Arg95, Leu96, Val97, Ala98, Phe99, Phe102, Gly103, Leu106, Trp137, Ser141, Glu146, Phe147, Thr148, Ala149, Leu150, Tyr 151 and Gly152, as set forth in TABLE 1.

38. An analysis method, executed by a computer system, for comparing the ability of a chemical entity to associate with a first molecule or molecular complex comprising a first active site and the ability of the chemical entity to associate with a second molecule or molecular complex comprising a second active site, the method comprising the steps of: (a) generating a model of the first active site using structure coordinates wherein the root mean square deviation between the structure coordinates and the structure coordinates of the Bcl-w amino acid residues defining a Bcl-w active site is not more than about 1.1 A, wherein the Bcl-w active site is defined by the structure coordinates of at least three Bcl-w amino acid residues selected from the group consisting of: Glu52, Arg56, Arg58, Glu85, Arg95 and Lysl 13, as set forth in TABLE 1; (b) performing a first fitting operation between the chemical entity and the model of the first active site; (c) quantifying the association between the chemical entity and the first active site model, based on the output of the first fitting operation; (d) performing a second fitting operation between the chemical entity and a model of the second active site; (e) quantifying the association between the chemical entity and the second active site model, based on the output of the second fitting operation; and (f) comparing the respective associations of the chemical entity with the first active site model and with the second active site model.

39. An analysis method according to claim 38, wherein the active site is further defined by the structure coordinates of at least three Bcl-w amino acid residues selected from the group consisting of: Glu52, Arg56, Arg58, Glu85, Arg95 and Lysl 13 as set forth in TABLE 1.

40. An analysis method according to claim 38, wherein the active site is defined by the structure coordinates of at least three Bcl-w amino acid residues selected from the group consisting of: Gln44, Ala45, Ala48, Ala49, Gly50, Glu52, Phe53, Arg56, Phe57, Arg58, Arg59, Asp63, Leu64, Ala66, Gln67, His69, Val70, Arg78, Phe79, Gln81, Val82, Ser83, Glu85, Leu86, Phe87, Gln88, Gly89, Gly90, Pro91, Asn92, Trp93, Gly94, Arg95, Val97, Phe99, Phe102, Leu106, Phe147, Thr148, Ala149, Leu150, Tyr151 and Gly152, as set forth in TABLE 1.

41. An analysis method according to claim 38, wherein the active site is defined by the structure coordinates of at least three Bcl-w amino acid residues selected from the group consisting of: Gln44, Ala45, Met46, Arg47, Ala48, Ala49, Gly50, Asp51, Glu52, Phe53, Glu54, Thr55, Arg56, Phe57, Arg58, Arg59, Thr60, Ser62, Asp63, Leu64, Ala65, Ala66, Gln67, Leu68, His69, Val70, Thr71, Ala75, Gln76, Gln77, Arg78, Phe79, Thr80, Gln81, Val82, Ser83, Asp84, Glu85, Leu86, Phe87, Gln88, Gly89, Gly90, Pro91, Asn92, Trp93, Gly94, Arg95, Leu96, Val97, Ala98, Phe99, Phe102, Gly103, Leu106, Trp137, Ser141, Glu146, Phe147, Thr148, Ala149, Leu150, Tyr151 and Gly152, as set forth in TABLE 1.

42. An analysis method according to claim 38, wherein the second molecule or molecular complex comprises an active site of another pro-survival protein.

43. An analysis method according to claim 42, wherein the other pro-survival protein is selected from the group consisting of: Bcl-2, Bcl-x_{L}, Mcl-1 and A1, and a variant thereof.

44. An analysis method, executed by a computer system, for identifying a chemical entity that associates with both a first molecule or molecular complex comprising a first active site and a second molecule or molecular complex comprising a second active site, the method comprising the steps o£ (a) generating a model of the first active site using structure coordinates wherein the root mean square deviation between the structure coordinates and the structure coordinates of the Bcl-w amino acid residues defining a Bcl-w active site is not more than about 1.1 A, wherein the Bcl-w active site is defined by the structure coordinates of at least three Bcl-w amino acid residues selected from the group consisting of: Glu52, Arg56, Arg58, Glu85, Arg95 and Lys113, as set forth in TABLE 1; (b) performing a fitting operation between the chemical entity and the model of the first active site; (c) quantifying the association between the chemical entity and the first active site model, based on the output of the first fitting operation; (d) performing a second fitting operation between the chemical entity and a model of the second active site; (e) quantifying the association between the chemical entity and the second active site model, based on the output of the second fitting operation; and (f) comparing the respective associations of the chemical entity with the first active site model and with the second active site model to determine whether the chemical entity associates individually with both the first molecule or molecular complex and the second molecule or molecular complex.

45. An analysis method, executed by a computer system, for identifying a chemical entity that associates more favourably with a first molecule or molecular complex comprising a first active site than with a second molecule or molecular complex comprising a second active site, the method comprising the steps of: (a) generating a model of the first active site using structure coordinates wherein the root mean square deviation between the structure coordinates and the structure coordinates of the Bcl-w amino acid residues defining a Bcl-w active site is not more than about 1.1 fir, wherein the Bcl-w active site is defined by the structure coordinates of at least three Bcl-w amino acid residues selected from the group consisting of: Glu52, Arg56, Arg58, Glu85, Arg95 and Lys 113, as set forth in TABLE 1; (b) performing a fitting operation between the chemical entity and the model of the first active site; (c) quantifying the association between the chemical entity and the first active site model, based on the output of the first fitting operation; (d) performing a second fitting operation between the chemical entity and a model of the second active site; (e) quantifying the association between the chemical entity and the second active site model, based on the output of the second fitting operation; and (f) comparing the respective associations of the chemical entity with the first active site model and with the second active site model to determine whether the chemical entity associates more favourably with the first molecule or molecular complex than with the second molecule or molecular complex.

46. An analysis method according to claim 44 or claim 45, wherein the active site is further defined by the structure coordinates of at least three Bcl-w amino acid residues selected from the group consisting of: Glu52, Arg56, Arg58, Glu85, Arg95 and Lys 113 as set forth in TABLE 1.

47. An analysis method according to claim 44 or claim 45, wherein the active site is defined by the structure coordinates of at least three Bcl-w amino acid residues selected from the group consisting of: Gln44, Ala45, Ala48, Ala49, Gly50, Glu52, Phe53, Arg56, Phe57, Arg58, Arg59, Asp63, Leu64, Ala66, Gln67, His69, Val70, Arg78, Phe79, Gln81, Val82, Ser83, Glu85, Leu86, Phe87, Gln88, Gly89, Gly90, Pro91, Asn92, Trp93, Gly94, Arg95, Val97, Phe99, Phe102, Leu106, Phe147, Thr148, Ala 149, Leu150, Tyr 151 and Gly 152, as set forth in TABLE 1.

48. An analysis method according to claim 44 or claim 45, wherein the active site is defined by the structure coordinates of at least three Bcl-w amino acid residues selected from the group consisting of: Gln44, Ala45, Met46, Arg47, Ala48, Ala49, Gly50, Asp51, Glu52, Phe53, Glu54, Thr55, Arg56, Phe57, Arg58, Arg59, Thr60, Ser62, Asp63, Leu64, Ala65, Ala66, Gln67, Leu68, His69, Val70, Thr71, Ala75, Gln76, Gln77, Arg78, Phe79, Thr80, Gln81, Val82, Ser83, Asp84, Glu85, Leu86, Phe87, Gln88, Gly89, Gly90, Pro91, Asn92, Trp93, Gly94, Arg95, Leu96, Val97, Ala98, Phe99, Phe102, Gly103, Leu106, Trp137, Ser141, Glu146, Phe147, Thr148, Ala149, Leu150, Tyr151 and Gly152, as set forth in TABLE 1.

49. A method for identifying a potential antagonist of a molecule comprising a Bcl-w-like active site, comprising the steps of: (a) generating a three-dimensional structure of the molecule comprising the active site using the atomic coordinates of at least three Bcl-w amino acid residues selected from the group consisting of: Arg59, Asp63, Leu64, Gln67, Phe79, Val 82, Val 102 and Leu106 as set forth in TABLE 1 ± a root mean square deviation from the Ca atoms of those residues of not more than 1.1 A; (b) employing the three-dimensional structure to identify, design or select the potential antagonist; (c) synthesising or otherwise obtaining the antagonist; and (d) contacting the antagonist with the molecule to determine the ability of the potential antagonist to interact with said molecule.

50. A method according to claim 49, wherein the three-dimensional structure of the molecule comprising the active site is generated further using structure coordinates of at least three Bcl-w amino acid residues selected from the group consisting of: Glu52, Arg56, Arg58, Asp63, Glu85, Arg95 and Lys 113 as set forth in TABLE 1±a root mean square deviation from the Ca atoms of those residues of not more than 1.1 A.

51. A method according to claim 49, wherein the three-dimensional structure of the molecule comprising the active site is generated further using structure coordinates of at least three Bcl-w amino acid residues selected from the group consisting of: Gln44, Ala45, Ala48, Ala49, Gly50, Glu52, Phe53, Arg56, Phe57, Arg58, Arg59, Asp63, Leu64, Ala66, Gln67, His69, Val70, Arg78, Phe79, Gln81, Val82, Ser83, Glu85, Leu86, Phe87, Gln88, Gly89, Gly90, Pro91, Asn92, Trp93, Gly94, Arg95, Val97, Phe99, Phe102, Leu106, Phe147, Thr148, Ala149, Leu150, Tyr151 and Gly152, as set forth in TABLE 1.

52. A method according to claim 49, wherein the three-dimensional structure of the molecule comprising the active site is generated further using structure coordinates of at least three Bcl-w amino acid residues selected from the group consisting of: Gln44, Ala45, Met46, Arg47, Ala48, Ala49, Gly50, Asp51, Glu52, Phe53, Glu54, Thr55, Arg56, Phe57, Arg58, Arg59, Thr60, Ser62, Asp63, Leu64, Ala65, Ala66, Gln67, Leu68, His69, Val70, Thr71, Ala75, Gln76, Gln77, Arg78, Phe79, Thr80, Gln81, Val82, Ser83, Asp84, Glu85, Leu86, Phe87, Gln88, Gly89, Gly90, Pro91, Asn92, Trp93, Gly94, Arg95, Leu96, Val97, Ala98, Phe99, Phe102, Gly103, Leu106, Trp137, Ser141, Glu146, Phe147, Thr148, Ala149, Leu150, Tyr151 and Gly152, as set forth in TABLE 1.

53. A method according to claim 49, wherein the three-dimensional structure of the molecule comprising the active site is created using the structure coordinates of all the Bcl-w amino acid residues as set forth in TABLE 1 ± a root mean square deviation from the Ca atoms of those residues of not more than 1.1 A.

54. An agent or antagonist designed or selected using a method according to claims 34 or 38.

55. A method for determining at least a portion of the three-dimensional structure of a molecule or molecular complex which contains at least some features that are structurally similar to Bcl-w by using at least some of the structural coordinates obtained for Bcl-w, the method comprising the steps o£ (a) obtaining crystals or a solution of the molecule or molecular complex whose structure is unknown; (b) generating X-ray diffraction data from the crystallised molecule or molecular complex and/or generating NMR data from the solution of the molecule or molecular complex; (c) comparing the data so generated with the solution coordinates or three dimensional structure of a Bcl-w derivative as set forth in TABLE 1, and (d) modeling the three dimensional structure of the unknown molecule or molecular complex on the basis of the Bcl-w derivative structure.

56. An agent or antagonist designed or selected using a method according to claims 44, 45 or 49.
